(19) United States
(12) Patent Application Publication
(10) Pub. No.: US 2012/0108512 A1
(43) Pub. Date: May 3, 2012

(54) STABLE PHARMACEUTICAL COMPOSITIONS OF PEPTIDE DERIVATIZED USING AN OXIME LINKER

(75) Inventor: Mats Reslow, Lund (SE)

(73) Assignee: Novo Nordisk Health Care AG, Zurich (CH)

(21) Appl. No.: 13/318,865

(22) PCT Filed: May 7, 2010
(86) PCT No.: PCT/EP2010/056261

§ 371 Date: Jan. 10, 2012

Related U.S. Application Data
(60) Provisional application No. 61/185,657, filed on Jun. 10, 2009.
(60) Provisional application No. 61/260,237, filed on Nov. 11, 2009.

(30) Foreign Application Priority Data
May 7, 2009 (EP) 091596585
Nov. 6, 2009 (EP) 091752519

(51) Int. Cl. A61K 38/27; A61P 5/06; C07K 14/61

(52) U.S. Cl. 514/11.4; 530/399

(57) ABSTRACT

The invention relates to stable pharmaceutical compositions comprising a therapeutic peptide derivatized with a property-modifying group, wherein the property-modifying group is conjugated to the peptide by use of a linker comprising an oxime bond.

### FIELD OF THE INVENTION

The invention relates to pharmaceutical compositions of therapeutic peptides, which peptides have been conjugated to a property-modifying group and where the property-modifying group is conjugated to the peptide by means of an oxime linkage.

### BACKGROUND OF THE INVENTION

In recent years, there has been a lot of focus on the use of biological peptides, often human peptides, for development of new therapeutics. The cloning of the human genome and the rapid development in the recombinant technology has helped enable a swift and easy access to such peptides. However, for a therapeutic peptide, there is often a desire to moderate the biological profile of the peptide, for instance making it resistant to fast hydrolysis, making it able to target specific tissues, coupling it physically with other biologically functional peptides, increasing the efficacy, abolishing some functionality etc. This can be achieved for instance by introducing specific mutations into the peptide creating peptide analogues or by derivatizing the peptide by conjugation of the peptide to so-called property modifying groups thereby creating peptide derivatives, or by a combination thereof creating derivatized peptide analogues (peptide analogue derivatives).

Such mutations, and particularly such derivatizations, may in some cases alter the stability of the peptide in pharmaceutical compositions as compared to pharmaceutical compositions of the peptide itself. For the convenience for the patient it is highly desirable that the pharmaceutical end-product is a ready-to-use solution, so a pharmaceutical composition of such peptides should be performed with these considerations in mind. Liquid formulations are generally preferred due to the convenience of manufacturing and use. However, peptide drugs may not be stable enough to be handled as a liquid formulation. Dried formulations, for instance lyophilised formulation have been successfully used to overcome stability problems. For derivatized peptides, the conjugation stability must also be considered.

For peptides derivatized with a property-modifying group, where the property-modifying group is conjugated to the peptide by use of a linker comprising an oxime bond, the presence of the oxime bond may provide challenges for the development of a ready-to-use, or liquid, solution. As an example, human growth hormone (hGH) may be chemical modified with property-modifying group such as a poly(ethylene glycol) (PEG) molecule, which effectively reduce renal clearance due to increased hydrodynamic volume of the modified hGH, which in turn increases the half-life of hGH and thereby decreasing the frequency of dosing. Generally, PEG molecules are connected to the peptide via a reactive group found on the peptide. Amino groups, such as lysine or N-terminus are normally used for such attachments. However, problems with controlling the conjugation chemistry due to the number of lysine residues present in human growth hormone results in a heterogeneous population of PEGylated hGH, which is disadvantageous for reproducibility and characterization of the therapeutic product. This problem may be solved for instance by site selective transglutaminase mediated conjugation of PEG on Gln-40 or Gln-141 in hGH, which for example results in the conjugating moiety is bounded to the hGH by means of linker comprising an oxime linkage (WO2005/070468, WO2006/134148).

Appropriate formulation is crucial in order to obtain a stable liquid formulation of peptides derivatized with a property-modifying group, particularly when the property-modifying group is conjugated to the peptide by use of for instance a linker comprising an oxime bond.

WO1996/41813 describes functionalized polymers comprising an organic polymer covalently attached to an amino-oxy oxime-forming group.

WO2005/035553 describes C-terminally conjugated peptides, such as hGH, wherein the linker may comprise an oxime linkage, and formulations thereof.

WO2007/025988 is concerned with liquid formulations of N-terminally oxime-pegylated hGH, wherein pH is 7 or below 7.

WO2005/070468 and WO2006/134148 are concerned with in-chain conjugated peptides, such as hGH, wherein the linker comprises an oxime linkage, and formulations thereof.

### SUMMARY OF THE INVENTION

The present invention provides an aqueous pharmaceutical compositions comprising a therapeutic peptide derivatized with a property-modifying group, wherein the property-modifying group is conjugated to the peptide by use of a linker comprising an oxime bond, and wherein the pH of the composition is above 7, such as from about 7.2 to about 9.

The present invention provides an aqueous pharmaceutical composition comprising a therapeutic peptide derivatized with a property-modifying group, wherein the property-modifying group is conjugated to the peptide by use of a linker comprising an oxime bond, and a buffer, wherein the buffer is citrate-NaOH or Tris-HCl.

The present invention provides an aqueous pharmaceutical composition comprising a therapeutic peptide derivatized with a property-modifying group, wherein the property-modifying group is conjugated to the peptide by use of a linker comprising an oxime bond, and a buffer, wherein the buffer is present in a concentration of no more than 100 mM.

The present invention provides an aqueous pharmaceutical compositions comprising a therapeutic peptide derivatized with a property-modifying group, wherein the property-modifying group is conjugated to the peptide by use of a linker comprising an oxime bond, and wherein the pH of the composition is above 7, such as from about 7.2 to about 9, wherein the property-modifying group is conjugated to the peptide in a position different from the N-terminal amino acid.

### DESCRIPTION OF THE DRAWINGS

FIG. 1. SEC purity of PEG-(Gln-141) hGH after incubation in different buffers at 40° C. for 1 week.

FIG. 2. Arrhenius plot of the rate constants from Table 1.

FIG. 3. Predicted oxime-linker degradation per year for liquid PEG-(Gln-141) hGH in different formulations.

FIG. 4. Effect of concentration of Tris on oxime-linker stability.

FIG. 5. Effect of buffer species (pH 7.5) on depegylation.

### DESCRIPTION OF THE INVENTION

The present invention provides a ready-to-use, aqueous pharmaceutical composition comprising a therapeutic peptide derivatized with a property-modifying group, wherein the property-modifying group is conjugated to the peptide by use of a linker comprising an oxime bond.

It has surprisingly been found that the nature of the buffer species, the pH, and the buffer concentration strongly influence the stability of the oxime bond and consequently the conjugation stability.

The present invention provides a pharmaceutical composition comprising a therapeutic peptide derivatized with a property-modifying group, wherein the property-modifying group is conjugated to the peptide by use of a linker comprising an oxime bond, and a buffer, wherein the buffer is selected from the group consisting of citrate-KOH, Tris-HNO₃, Tris-H₂PO₄, citrate-NaOH or Tris-HCl. In one embodiment, the buffer is selected from citrate-NaOH or Tris-HCl.

The present invention also provides a pharmaceutical composition comprising a therapeutic peptide derivatized with a property-modifying group, wherein the property-modifying group is conjugated to the peptide by use of a linker comprising an oxime bond, and a buffer, wherein the buffer is present in a concentration of from 1 mM to 100 mM, for instance from 5 mM to 100 mM, such as from 10 mM to 100 mM, for instance from 25 mM to 100 mM, such as from 50 to 100 mM. In one embodiment, the buffer is present in a concentration of from 1 mM to 50 mM, for instance from 5 mM to 50 mM, such as from 10 mM to 50 mM, for instance from 25 mM to 50 mM. In one embodiment, the buffer is present in a concentration of from 1 mM to 25 mM, for instance from 5 mM to 25 mM, such as from 10 mM to 25 mM. In one embodiment, the buffer is present in a concentration of from 1 mM to 20 mM, for instance from 5 mM to 20 mM, such as from 10 mM to 20 mM. In one embodiment, the buffer is present in a concentration of from 1 mM to 10 mM, for instance from 5 mM to 10 mM, such as 5 mM.

The present invention provides a pharmaceutical composition comprising a therapeutic peptide derivatized with a property-modifying group, wherein the property-modifying group is conjugated to the peptide by use of a linker comprising an oxime bond, wherein the pH of the composition is from about 7.2 to about 9, for instance from about 7.2 to about 8, such as from about 7.2 to about 7.7, for instance about 7.2.

The term “peptide” is intended to indicate a sequence of two or more amino acids joined by peptide bonds, wherein said amino acids may be natural or unnatural. The term encompasses the terms polypeptides and proteins, which may consist of two or more polypeptides held together by covalent interactions, such as for instance cysteine bridges, or non-covalent interactions. The term “peptide” includes any suitable peptide and may be used synonymously with the terms polypeptide and protein, unless otherwise stated or contradicted by context; provided that the reader recognize that each type of respective amino acid polymer-containing molecule may be associated with significant differences and thereby form individual embodiments of the present invention (for example, a peptide such as an antibody, which is composed of multiple polypeptide chains, is significantly different from, for example, a single chain antibody, a peptide immunoadhesin, or single chain immunogenic peptide). The term “polypeptide” herein should generally be understood as referring to any suitable polypeptide of any suitable size and composition (with respect to the number of amino acids and number of associated chains in a peptide molecule). Moreover, peptides in the context of the inventive methods and compositions described herein may comprise non-naturally occurring and/or non-L amino acid residues, unless otherwise stated or contradicted by context. Non-limiting examples of such amino acid residues include for instance 2-aminoadipic acid, 3-aminoadipic acid, β-alanine, β-aminopropionic acid, 2-amino-butyric acid, 4-aminobutyric acid, 6-aminocaproic acid, 2-aminoheptanoic acid, 2-amino-isobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethyl-asparagine, hydroxylysine, allohydroxylysine, 3-hydroxyproline, 4-hydroxyproline, iso-desmosine, alloisoleucine, N-methylglycine, N-methylisoleucine, 6-N-methyllysine, N-methyl-valine, norvaline, norleucine, ornithine, and statine halogenated amino acids.

In one embodiment, the peptide comprises at least one asparagine amino acid residue or at least one glutamine amino acid residue, wherein said amino acid residue is not derivatized with the property-modifying group. This may make the peptide susceptible to deamidation, which is a chemical reaction in which an amide functional group is removed from an organic compound. In biochemistry, the reaction is important in the degradation of proteins because it damages the amide-containing side chains of the amino acids asparagine and glutamine. The rate of deamidation increases with pH, the higher the pH, the higher the reaction rate of deamidation. Deamidation can interfere with activity of peptides, but it may also simply result in a heterogenous population of peptides (with different level of deamidation), where activity is retained in all or some peptides regardless of the amount of deamidation.

In one embodiment, the derivatized therapeutic peptide is a derivatized growth hormone compound. In the present context, “growth hormone compound” is intended to indicate a peptide which exhibits growth hormone activity as determined in Assay I herein (see Example 3). In one embodiment, the (underivatized) growth hormone compound exhibits an activity above 10%, such as above 20%, such as above 40%, such as above 60%, such as above 80% of that of hGH (the sequence of which is presented as SEQ ID No. 1) in said assay. The activity of the derivatized growth hormone can vary significantly depending on the properties that the property-modifying group confers on the peptide. For instance, if the property-modifying group confers increased half-life, then the activity of the derivatized growth hormone can be considerably less than the activity of hGH, because the prolonged half-life may make up for a substantial amount of the reduction of activity.

In one embodiment, the derivatized growth hormone is present in the pharmaceutical composition in a concentration from 1-10 mg (underivatized growth hormone)/ml. In one embodiment, the derivatized growth hormone is present in the pharmaceutical composition in a concentration from 2-8 mg (underivatized growth hormone)/ml. In one embodiment, the derivatized growth hormone is present in the pharmaceutical composition in a concentration from 3-7 mg (underivatized growth hormone)/ml.

In one embodiment, the growth hormone compound has at least 80%, such as at least 85%, such as at least 90%, such as at least 95%, such as at least 97%, such as at least 98%, or such as at least 99% identity with hGH (SEQ ID No. 1).

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percentage of identical matches between two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA, 89:10915-10919 (1992) for the BLOSUM 62 comparison matrix) is also used by the algorithm.

Preferred parameters for a peptide sequence comparison include the following: Algorithm: Needleman et al., J. Mol. Biol, 48, 443-453 (1970); Comparison matrix: BLOSUM 62 from Henikoff et al., Proc. Natl. Acad. Sci. USA, 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, the derivatized therapeutic peptide is a derivatized hGH.

The therapeutic peptide for use in a pharmaceutical composition according to the present invention is derivatized with a property-modifying group (resulting in a so-called conjugate), wherein the property-modifying group is conjugated to the peptide by use of a linker comprising an oxime bond.

The term “conjugate” or “derivative” as a noun is intended to indicate a modified peptide, i.e. a peptide with a moiety bonded to it to modify the properties of said peptide. As verbs, the terms are intended to indicate the process of bonding a moiety to a peptide to modify the properties of said peptide. The words “conjugate” and “derivatize” are used interchangeably herein when describing a conjugated or derivatized peptide. A conjugated or derivatized peptide should generally be understood as referring to a peptide in which one or more of the amino acid residues of the peptide have been chemically modified (for instance by alkylation, acylation, ester formation, or amide formation) or associated with one or more non-amino acid organic and/or inorganic atomic or molecular substituents (for instance water-soluble polymers, such as a polyethylene glycol (PEG) group, a lipophilic substituent (which optionally may be linked to the amino acid sequence of the peptide by a spacer residue or group such as β-alanine, γ-aminobutyric acid (GABA), L/D-glutamic acid, succinic acid, and the like), a fluorophore, biotin, a radionuclide, etc.). peptides, which are therapeutically useful and where therapy using the peptides would benefit from for instance an increased retention time, are particularly suitable for use in a method of the present invention. A “derivatized amino acid residue” designates an amino acid residue to which a property-modifying group has been attached.

In one embodiment, the property-modifying group is conjugated to the peptide in an in-chain position, that is a position that is different from the N-terminal amino acid. In one embodiment, the property-modifying group is conjugated to the peptide in an in-chain position, that is a position that is different from the C-terminal amino acid. In one embodiment, the property-modifying group is conjugated to the peptide in an in-chain position, that is a position that is different from the N-terminal amino acid and different from the C-terminal amino acid.

The term “property-modifying group” is intended to indicate a chemical group, which, when attached to the peptide in question alters one or more of the physicochemical or pharmacological properties of the peptide. Such properties could be solubility, tissue- and organ distribution, lipophilicity, susceptibility to degradation by various proteases, affinity to plasma proteins, such as albumin, functional in vivo half-life, plasma in vivo half-life, mean residence time, clearance, immunogenicity, and renal filtration. It is well-known in the art, that several types of chemical groups may have such property-modifying effects.

In one embodiment, the property-modifying group is a water-soluble polymer. In one embodiment, the property-modifying group is a PEG group. In one embodiment, the property-modifying group is an mPEG group.

The term “polyethylene glycol”, “Peg” or “PEG” (poly(ethylene glycol) means a polydisperse or monodisperse diradical of the structure

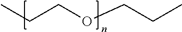

wherein n is an integer larger than 1, and its molecular weight is between approximately 100 and approximately 1,000,000 Da.

The term “mPEG” or “mPeg” means a polydisperse or monodisperse radical of the structure

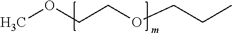

wherein m is an integer larger than 1. Thus, an mPEG wherein m is 90 has a molecular weight of 3991 Da, i.e. approximately 4 kDa. Likewise, an mPEG with an average molecular weight of 20 kDa has an average m of 454. Due to the process for producing mPEG these molecules often have a distribution of molecular weights. This distribution is described by the polydispersity index.

Due to this distribution of m, mPEG with a molecular weight of 20 kDa may also be referred to as MeO-(CH₂CH₂O)₄₀₀₋₅₀₀, mPEG with a molecular weight of 30 kDa may also be referred to as MeO-(CH₂CH₂O)₆₀₀₋₇₀₀, and mPEG with a molecular weight of 40 kDa may also be referred to as MeO-(CH₂CH₂O)₈₅₀₋₉₅₀. The heavier mPEG chains may be difficult to prepare as a single chain molecule, and they are thus made as branched mPEG. Notably, mPEG with a molecular weight of 40 kDa may be achieved with as a branched mPEG comprising to arms of 20 kDa each.

The term “polydispersity index” as used herein means the ratio between the weight average molecular weight and the number average molecular weight, as known in the art of polymer chemistry (see for instance “Polymer Synthesis and Characterization”, J. A. Nairn, University of Utah, 2003). The polydispersity index is a number which is greater than or equal to one, and it may be estimated from Gel Permeation Chromatographic data. When the polydispersity index is 1, the product is monodisperse and is thus made up of compounds with a single molecular weight. When the polydispersity index is greater than 1 it is a measure of the polydispersity of that polymer, i.e. how broad the distribution of polymers with different molecular weights is.

The use of for example “mPEG20000” in formulas, compound names or in molecular structures indicates an mPEG residue wherein mPEG is polydispersed and has a molecular weight of approximately 20 kDa.

The polydispersity index typically increases with the molecular weight of the PEG or mPEG. When reference is made to 20 kDa PEG and in particular 20 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 30 kDa PEG and in particular 30 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 40 kDa PEG and in particular 40 kDa mPEG it is intended to indicate a compound (or in fact a mixture of compounds) with a polydisperisty index below 1.06, such as below 1.05, such as below 1.04, such as below 1.03, such as between 1.02 and 1.03.

In one embodiment, the peptide derivatized with a property-modifying group is a peptide as described in WO2005/070468 and WO2006/134148. In short, such a peptide may be represented by formula [V]

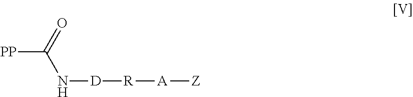

wherein PP represents a radical of the therapeutic peptide comprising a glutamine residue, to which the property-modifying group is attached, wherein said radical formally obtained by removing —C(═O)—NH₂ from the side chain of a glutamine residue present in the peptide;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂— or C₅₋₁₅heteroalkylene;
A represents an oxime bond;
Z represents said property-modifying group.

The term “oxime bond” is intended to indicate a chemical substructure of the structure —O—N═. In the structural formulas used herein, the oxime bond represented by A in the formula may be positioned in either direction, that is either —O—N═ or ═N—O—. In one embodiment, the direction of the oxime bond in the structural formula given is —O—N═. In one embodiment, the direction of the oxime bond in the structural formula given is ═N—O—.

In one embodiment the therapeutic peptide derivative is a growth hormone compound, wherein the glutamine residue is present in-chain in the growth hormone peptide.

In one embodiment the therapeutic peptide derivative is a growth hormone compound, where in the glutamine residue is a naturally occurring glutamine present in a position corresponding to Gln40 or Gln141 in human growth hormone.

A therapeutic peptide derivatized with a property-modifying group, wherein the property-modifying group is conjugated to the peptide by use of a linker comprising an oxime bond, may be prepared in a number of ways. One way is to contact a property modifying group bearing an aminoxy functionality with a peptide bearing an aldehyde or ketone group. A second way is to contact a peptide bearing an aminoxy functionality with a property modifying group bearing an aldehyde or ketone

In one embodiment, a therapeutic peptide derivatized with a property-modifying group for use in a pharmaceutical composition according to the invention, wherein the property-modifying group is conjugated to the peptide by use of a linker comprising an oxime bond, and which peptide comprises a glutamine residue, who which the property-modifying group is to be conjugated, is prepared as described in WO2005/070468 and WO2006/134148. In short, said method comprise reacting in one or more steps such glutamine residue comprising polypeptide represented by formula [I]

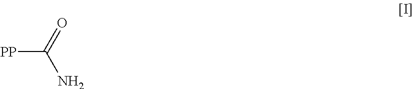

wherein PP represents a polypeptide radical obtained by removing —C(═O)—NH₂ from the side chain of a glutamine residue present in the polypeptide, with a nitrogen containing nucleophile of formula [II]

H₂N-D-R—X [II]

wherein D represents —O— or a single bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
X represents —O—NH₂, an aldehyde, a ketone, or a latent group which upon further reaction may be transformed into —O—NH₂, an aldehyde or a ketone;
in the presence of transglutaminase to form a transaminated polypeptide of formula [III]

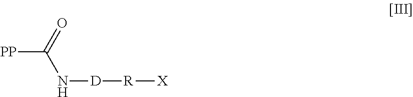

optionally, if X is a latent group, transforming said latent group into —O—NH₂, an aldehyde or a ketone,
said transaminated polypeptide being further reacted with a second compound of formula [IV]

Y—Z [IV]

wherein Y,
if X represents an aldehyde, a ketone, or a latent group which upon further reaction may be transformed an aldehyde or a ketone, represents —O—NH₂; or,
if X represents —O—NH₂, or a latent group which upon further reaction may be transformed into —O—NH₂, represents an aldehyde or a ketone; and
Z represents a moiety selected amongst

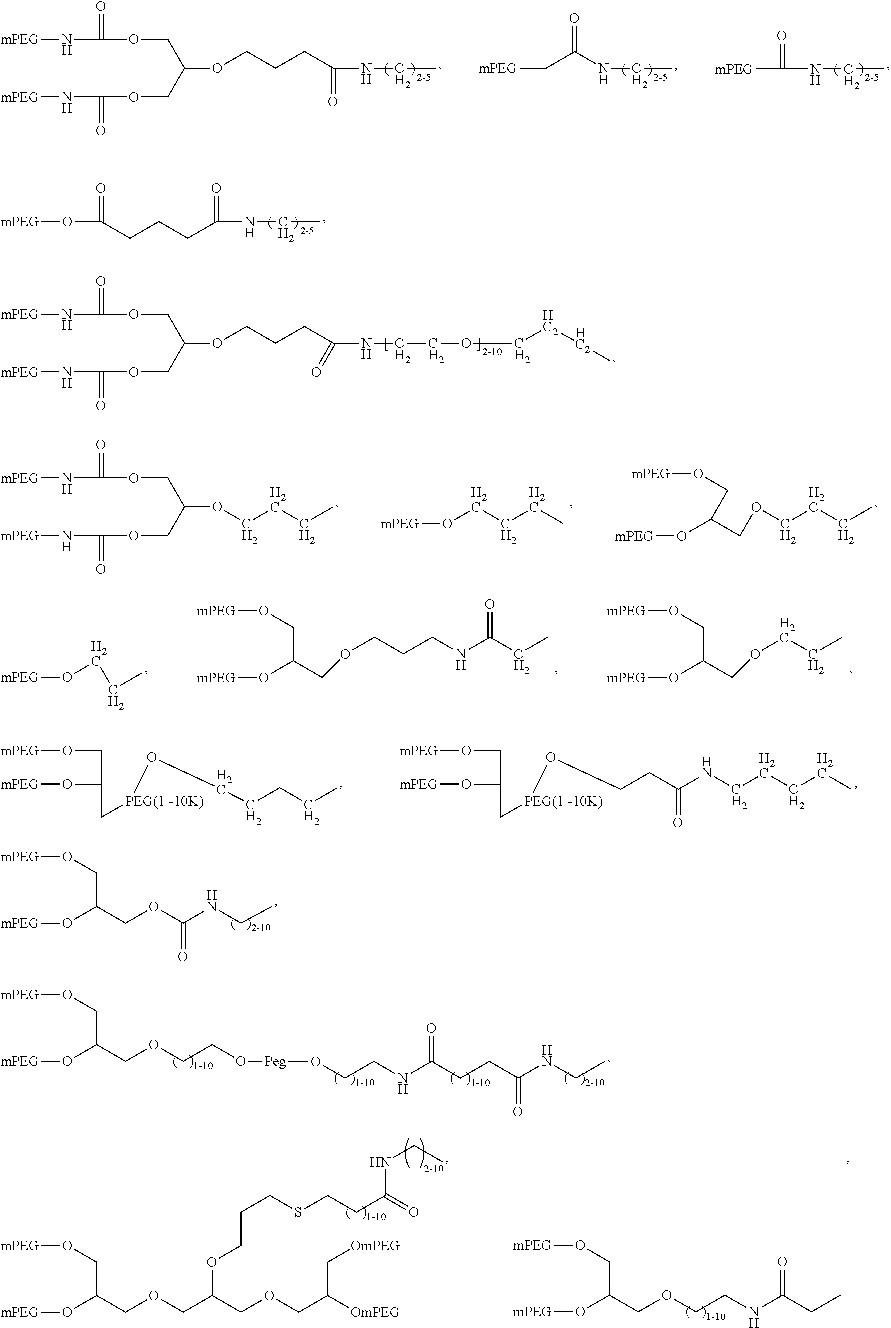

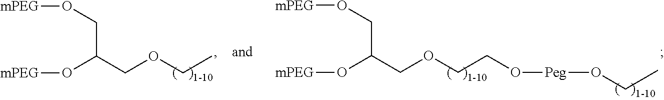

wherein, unless otherwise indicated, mPEG indicates a mPEG with a molecular weight of between 5 kDa and 40 kDa and PEG indicates a PEG with a molecular weight between 1 kDa and 10 kDa;
provided that if Z is

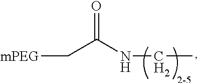

then PEG is 10 kDa PEG to form a PEGylated polypeptide of formula [V]

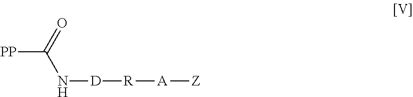

wherein A represents an oxime bond.

In one embodiment, the pharmaceutical composition according to the present invention additionally comprises a pharmaceutically acceptable preservative. In one embodiment, the preservative is selected from the group consisting of phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof. In one embodiment, the preservative is phenol. In one embodiment, the preservative is present in a concentration from about 0.1 mg/ml to about 20 mg/ml. In one embodiment, the preservative is present in a concentration of from about 0.1 mg/ml to about 10 mg/ml. In one embodiment, the preservative is present in a concentration of from about 10 mg/ml to about 20 mg/ml. In one embodiment, the preservative is present in a concentration of from about 5 mg/ml to about 10 mg/ml. In one embodiment, the preservative is present in a concentration of from about 0.1 mg/ml to about 5 mg/ml, such as for instance from about 1.0 mg/ml to 5 mg/ml, such as in a concentration of 1.0, 1.5, 2.0, 2.5, 3.0, 3.5, 4.0, 4.5, or 5.0 mg/ml. In one embodiment, the preservative is present in a concentration of from 4.0 to 5.0 mg/ml, such as in a concentration of 4.0, 4.5, or 5.0 mg/ml.

In one embodiment, the pharmaceutical composition additionally comprises an isotonic agent. In one embodiment, the isotonic agent is selected from the group consisting of a salt (for instance sodium chloride), a polyhydric alcohol (for instance propylenglycol, xylitol, mannitol, sorbitol or glycerol), a monosaccharide (for instance glucose or maltose), a disaccharide (for instance sucrose), an amino acid (e.g. L-glycine, L-histidine, arginine, lysine, isoleucine, aspartic acid, tryptophan, threonine), polyethyleneglycol (for instance PEG400), or mixtures thereof. In one embodiment, the isotonic agent is selected from the group consisting of sodium chloride, glycerol, mannitol, glucose, sucrose, 1-glycine, L-histidine, arginine, lysine or mixtures thereof. In one embodiment, the isotonic agent is mannitol. When the pharmaceutical composition comprises an isotonic agent, it is present in a concentration, which results in an isotonic pharmaceutical composition. In one embodiment, the isotonic agent is present in a concentration from 1 mg/ml to 50 mg/ml. In one embodiment, the isotonic is present in a concentration from 1 mg/ml to 7 mg/ml. In one embodiment, the isotonic agent is present in a concentration from 8 mg/ml to 16 mg/ml. In one embodiment, the isotonic agent is present in a concentration from 17 mg/ml to 50 mg/ml. In one embodiment, the isotonic agent is present in a concentration from 20 to 50 mg/ml. In one embodiment, the isotonic agent is present in a concentration from 30 to 50 mg/ml. In one embodiment, the isotonic agent is present in a concentration from 35 to 45 mg/ml. In one embodiment, the isotonic agent is present in a concentration from 40 to 50 mg/ml. In one embodiment, the isotonic agent is present in a concentration from 40 to 42 mg/ml. In one embodiment, the isotonic agent is present in a concentration of about 41 mg/ml, such as for instance 40.6, 40.7, 40.8, 40.9, 41.0, 41.1, 41.2, 41.3, or 41.4 mg/ml. In one embodiment, the isotonic agent is present in a concentration of about 41 to 42 mg/ml, such as for instance in a concentration of about 41.5 to 42 mg/ml, such as for instance 41.4, 41.6, 41.7, 41.8, 41.9 or 42.0 mg/ml.

It is to be understood that for the purpose of the present application the various embodiments described herein defining components, concentration limits, ph values and/or interval of the aqueous pharmaceutical composition using the wording “comprising” may be also applied in composition “consisting” of the defined components, concentration limits, ph values or interval unless otherwise stated or clearly contradicted by context.

In one embodiment the invention relates to an aqueous pharmaceutical compositions consisting of: a therapeutic peptide derivatized with a property-modifying group, wherein the property-modifying group is conjugated to the peptide by use of a linker comprising an oxime bond, a buffer, an isotonic agent and a preservative. In one further such embodiment the pH of the composition is above 7, such as from about 7.2 to about 8, and the property-modifying group is conjugated to the peptide in a position different from the N-terminal amino acid.

In one embodiment the aqueous pharmaceutical compositions consists of a therapeutic peptide derivatized with a property-modifying group, wherein the property-modifying group is conjugated to the peptide by use of a linker comprising an oxime bond in an in-chain position, a buffer providing a pH from about 7.2 to 8.0, an isotonic agent and a preservative. In one further such embodiment the isotonic agent is mannitol and the preservative is phenol.

In one embodiment the pharmaceutical compositions is stable for at least 6 months, such as at least 12 months, such as at least 18 months, such as at least 24 months, such as at least 30 months, such as at least 36 months e.g. the oxime-linkage is stable during prolonged storage at 5° C.

In one embodiment the pharmaceutical composition, wherein the property-modifying group is a PEG or mPEG group, said PEGylated peptide is stable for at least 6 months as the main cause of de-PEGylation is considered to be due to instability of the oxime-linkage.

In a further embodiment a pharmaceutical composition according to the invention comprises a minimum of dePEGylated compound, such as at most 5%, such as at most 4.5% or such as at most 4% after storage at 5° C. for such as 6 months, such as 12 months, such as 18 months, such as 24 months.

In a further embodiment the pharmaceutical composition comprising a PEGylated peptide is stable for at least 6 months, e.g. the amounts of de-PEGylation of said PEGylated peptide is at most 5%, such as at most 4.5%, 4.0%, 3.5%, 3.0%, 2.5%, 2.0% or such as at most 1.5% during storage at 5° C. for at least 6 months.

In a further embodiment de-PEGylation of said PEGylated peptide in the pharmaceutical composition is at most 5%, such as at most 4.5% or such as at most 4% or such as at most 2.8% or such as at most 2.6 or such as at most 2.4% or such as at most 2.2% or such as at most 2.0% during storage at 5° C. for such as at least 12 months, such as 18 months or such as 24 months.

The present invention also provides a pharmaceutical composition according to the present invention, wherein the therapeutic peptide is growth hormone, for use in therapy, such as in the treatment of diseases benefiting from an increase in the level of circulating growth hormone, such as growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; short stature due to glucocorticoid treatment in children; acceleration of the healing of muscle tissue, nervous tissue or wounds; the acceleration or improvement of blood flow to damaged tissue; or the decrease of infection rate in damaged tissue.

The present invention also provides the use of a pharmaceutical composition according to the present invention for treatment of diseases benefiting from an increase in the level of circulating growth hormone, such as those described above, as well as a method for treating a disease benefiting from an increase in the level of circulating growth hormone, such as the diseases described above, wherein said method comprising administration of a therapeutically effective amount of a pharmaceutical composition according to the present invention to a subject in need thereof.

The term “treatment” and “treating” is intended to indicate the management and care of a patient for the purpose of combating a condition, a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs.

The term “therapeutically effective amount” of a compound is intended to indicate an amount sufficient to cure, alleviate or partially arrest the clinical manifestations of a given disease and its complications. An amount adequate to accomplish this is defined as “therapeutically effective amount”. Effective amounts for each purpose will depend on for instance the severity of the disease or injury as well as the weight, sex, age and general state of the subject. It will be understood that determining an appropriate dosage may be achieved using routine experimentation, by constructing a matrix of values and testing different points in the matrix, which is all within the ordinary skills of a trained physician or veterinary.

In one embodiment, the compound of the invention is administered by parental administration. A typical parenteral dose is in the range of 10⁻⁹ mg/kg to about 100 mg/kg body weight per administration. Typical administration doses are from about 0.0000001 to about 10 mg/kg body weight per administration. The exact dose will depend on for instance indication, medicament, frequency and mode of administration, the sex, age and general condition of the subject to be treated, the nature and the severity of the disease or condition to be treated, the desired effect of the treatment and other factors evident to the person skilled in the art. Typical dosing frequencies are twice daily, once daily, bi-daily, twice weekly, once weekly or with even longer dosing intervals.

When the therapeutic peptide is a growth hormone compound, a typical dosing range could be from 10 μg/kg/week to 100 μg/kg/week, such as from 10 μg/kg/week to 50 μg/kg/week, for instance from 10 μg/kg/week to 45 μg/kg/week, such as 10 μg/kg/week, 15 μg/kg/week, 20 μg/kg/week, 25 μg/kg/week, 30 μg/kg/week, 35 μg/kg/week, 40 μg/kg/week, or 45 μg/kg/week, wherein the amount of the therapeutic peptide is measured as weight of underivatized growth hormone compound.

In one embodiment, where the therapeutic peptide is a growth hormone the dosing frequency is less than once daily, such as bi-daily, twice weekly, once weekly or with even longer dosing intervals. In one embodiment the pharmaceutical composition comprising a growth hormone conjugate is for administering twice weekly, once weekly or with a frequency below once weekly, such as each 10^{th} day or each second week.

In one embodiment where the therapeutic peptide is a growth hormone the pharmaceutical composition is for administering within a specified time interval on the days of administration, said time interval may be related to meal times or sleep.

In one such embodiment the pharmaceutical composition is for administration in the morning such as within 2 hours before or 2 hours after breakfast or such as within 1 hour before or 1 hour after breakfast. In a different embodiment the pharmaceutical composition is for administration during the day, such as within two hours before or two hours after lunch. In a different embodiment the pharmaceutical composition is for administration during the afternoon, such as at least 2 hours after lunch or more than 2 hours before the evening meal. In a different embodiment the pharmaceutical composition is for administration during the evening, such as within two hours before or two hours after the evening meal. In a further embodiment the pharmaceutical composition is for administration within 1 or 2 hours after waking-up in the morning or 1 or two hours before going to sleep in the evening.

In one embodiment said disease is selected from wasting in AIDS patients, GH-deficiency due to a pituitary tumour, and poor growth in children due to GH-deficiency, renal failure, Turner syndrome, and Prader-Willi syndrome.

As it can be seen in the following examples, pharmaceutical compositions according to the present invention provide superior stability of the oxime-linker.

For pharmaceutical compositions of growth hormone compounds derivatized with a property-modifying group, wherein the property-modifying group is conjugated to the peptide by use of a linker comprising an oxime bond, such as the derivatized growth hormone molecules described in WO2005/070468 and WO2006/134148, the pharmaceutical compositions according to the present invention is of particular value. Human growth hormone is known to degrade during storage. Main degradation products arise from deamidation at Asn-residues (Asn-149 and Asn-152). Stability studies of PEGylated human growth hormone has shown that the main degradation products are caused by similar deamidation events. The pharmaceutical compositions of the present invention minimizes this deamidation, while optimizing the stability of the oxime-linker, but any therapeutic peptide derivatized with a property-modifying group, wherein the property-modifying group is conjugated to the peptide by use of a linker comprising an oxime bond will benefit from the present invention.

The following is a none-limiting list of embodiments of the present invention.

Embodiment 1: An aqueous pharmaceutical composition comprising a therapeutic peptide derivatized with a property-modifying group, wherein the property-modifying group is conjugated to the peptide by use of a linker comprising an oxime bond, and a buffer, wherein the buffer is selected from the group consisting of citrate-KOH, Tris-HNO₃, Tris-H₂PO₄, citrate-NaOH or Tris-HCl.

Embodiment 2: An aqueous pharmaceutical composition according to embodiment 1, wherein the buffer is citrate-NaOH or Tris-HCl.

Embodiment 3: An aqueous pharmaceutical composition comprising a therapeutic peptide derivatized with a property-modifying group, wherein the property-modifying group is conjugated to the peptide by use of a linker comprising an oxime bond, and a buffer, wherein the buffer is present in a concentration of 100 mM or less.

Embodiment 4: A pharmaceutical composition according to embodiment 1 or embodiment 2, wherein the buffer is present in a concentration of from 1 mM to 100 mM.

Embodiment 5: A pharmaceutical composition according to embodiment 3 or embodiment 4, wherein the buffer is present in a concentration of from 1 mM to 50 mM.

Embodiment 6: A pharmaceutical composition according to embodiment 5, wherein the buffer is present in a concentration of from 1 mM to 25 mM.

Embodiment 7: A pharmaceutical composition according to embodiment 6, wherein the buffer is present in a concentration of from 1 mM to 20 mM.

Embodiment 8: A pharmaceutical composition according to any of embodiments 3 to 7, wherein the buffer is present in a concentration of 20 mM.

Embodiment 9: A pharmaceutical composition according to embodiment 7, wherein the buffer is present in a concentration of from 1 mM to 10 mM.

Embodiment 10: A pharmaceutical composition according to embodiment 9, wherein the buffer is present in a concentration of from 1 mM to 5 mM.

Embodiment 11: A pharmaceutical composition according to embodiment 10, wherein the buffer is present in a concentration of 5 mM.

Embodiment 12: A pharmaceutical composition according to any of embodiments 1 to 11, wherein the pharmaceutical composition further comprises a preservative.

Embodiment 13: A pharmaceutical composition according to embodiment 12, wherein said preservative is phenol.

Embodiment 14: A pharmaceutical composition according to embodiment 12 to embodiment 13, wherein said preservative is present in a concentration from 0.1 mg/ml to 20 mg/ml.

Embodiment 15: A pharmaceutical composition according to embodiment 14, wherein said preservative is present in a concentration from 1.0 mg/ml to 10 mg/ml.

Embodiment 16: A pharmaceutical composition according to embodiment 15, wherein said preservative is present in a concentration of from about 1.0 mg/ml to about 5.0 mg/ml.

Embodiment 17: A pharmaceutical composition according to embodiment 15, wherein said preservative is present in a concentration of from about 4.0 mg/ml to about 5.0 mg/ml.

Embodiment 18: A pharmaceutical composition according to any of embodiments 1 to 17, wherein the pharmaceutical composition is isotonic.

Embodiment 19: A pharmaceutical composition according embodiment 18, wherein said isotonicity are provided by the presence of an isotonic agent.

Embodiment 20: A pharmaceutical composition according to any of embodiments 1 to 17, wherein the pharmaceutical composition further comprises an isotonic agent.

Embodiment 21: A pharmaceutical composition according to embodiment 19 or embodiment 20, wherein said isotonic agent is mannitol.

Embodiment 22: A pharmaceutical composition according to any of embodiments 19 to 21, wherein said isotonic agent is present in a concentration from 1 mg/ml to 50 mg/ml.

Embodiment 23: A pharmaceutical composition according to any of embodiments 19 to 22, wherein said isotonic agent is present in a concentration of from about 40 mg/ml to 50 mg/ml.

Embodiment 24: A pharmaceutical composition according to any of embodiments 19 to 23, wherein said isotonic agent is present in a concentration of from about 40 mg/ml to about 42 mg/ml.

Embodiment 25: A pharmaceutical composition according to any of embodiments 19 to 24, wherein said isotonic agent is present in a concentration of about 41 mg/ml.

Embodiment 26: An aqueous pharmaceutical composition comprising a therapeutic peptide derivatized with a property-modifying group, wherein the property-modifying group is conjugated to the peptide by use of a linker comprising an oxime bond, wherein the pH of the composition is above 7.

Embodiment 27: A pharmaceutical composition according to any of embodiments 1 to 25, wherein the pH of the composition is above 7.

Embodiment 28: A pharmaceutical composition according to embodiment 26 or embodiment 27, wherein the pH of the composition is from about 7.2 to about 9.

Embodiment 29: A pharmaceutical composition according to embodiment 28, wherein the pH of the composition is from about 7.2 to about 8.

Embodiment 30: A pharmaceutical composition according to embodiment 29, wherein the pH of the composition is from about 7.2 to about 7.7.

Embodiment 31: A pharmaceutical composition according to embodiment 30, wherein the pH of the composition is from about 7.2 to about 7.5.

Embodiment 32: A pharmaceutical composition according to embodiment 31, wherein the pH of the composition is about 7.2.

Embodiment 33: A pharmaceutical composition according to any of embodiments 1 to 32, wherein the property-modifying group is conjugated to the peptide in a position different from the N-terminal amino acid.

Embodiment 34: A pharmaceutical composition according to any of embodiments 1 to 33, wherein the property-modifying group is conjugated to the peptide in a position different from the C-terminal amino acid.

Embodiment 35: A pharmaceutical composition according to any of embodiments 1 to 34, wherein the property-modifying group is a water-soluble polymer.

Embodiment 36: A pharmaceutical composition according to embodiment 35, wherein the property-modifying group is a PEG group.

Embodiment 37: A pharmaceutical composition according to embodiment 35, wherein the property-modifying group is a mPEG group.

Embodiment 38: A pharmaceutical composition according to any of embodiments 1 to 37, wherein the derivatized therapeutic peptide comprises at least one asparagine amino acid residue or at least one glutamine amino acid residue, wherein said amino acid residue is not derivatized with the property-modifying group.

Embodiment 39: A pharmaceutical composition according to any of embodiments 1 to 38, wherein the derivatized therapeutic peptide is a derivatized growth hormone compound.

Embodiment 40: A pharmaceutical composition according to Embodiment 39, wherein the property-modifying group is conjugated to the growth hormone compound in a position corresponding to Gln40 and/or Gln141 in human growth hormone.

Embodiment 41: A pharmaceutical composition according to Embodiment 39, wherein the property-modifying group is conjugated to the growth hormone compound in a position corresponding to Gln40 in human growth hormone.

Embodiment 42: A pharmaceutical composition according to Embodiment 39, wherein the property-modifying group is conjugated to the growth hormone compound in a position corresponding to Gln141 in human growth hormone.

Embodiment 43: A pharmaceutical composition according to any of the embodiments 39 to 42, wherein the growth hormone compound exhibits an activity above 10%, such as above 20%, such as above 40%, such as above 60%, such as above 80% of that of hGH in Assay I.

Embodiment 44: A pharmaceutical composition according to any of the embodiments 39 to 43, wherein the growth hormone compound has at least 80%, such as at least 85%, such as at least 90%, such as at least 95%, such as at least 97%, such as at least 98%, or such as at least 99% identity with SEQ ID No. 1.

Embodiment 45: A pharmaceutical composition according to any of embodiments 39 to 44, wherein the growth hormone compound is hGH.

Embodiment 46: A pharmaceutical composition according to any of embodiments 1 to 38, wherein the therapeutic peptide comprises at least one glutamine residue and wherein the derivatized therapeutic peptide is obtainable by, has been obtained by, or has been derivatized by use of a method for covalently attaching a property-modifying group to a peptide, wherein said method comprises reacting in one or more steps a peptide of formula [I]

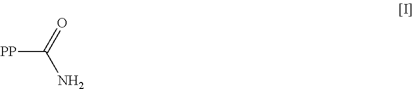

wherein PP represents a radical of the therapeutic peptide, which radical is formally obtained by removing —C(═O)—NH₂ from the side chain of a glutamine residue present in the peptide, with a nitrogen containing nucleophile of formula [II]

H₂N-D-R—X [II]

wherein D represents —O— or a single bond;
R represents C₁₋₆alkylene, C₅₋₁₅heteroalkylene, —(CH₂)₄—CH(NH₂)—CO—NH—CH₂—, or (CH₂)₄—CH(NHCOCH₃)—CO—NH—CH₂—;
X represents —O—NH₂, an aldehyde, a ketone, or a latent group which upon further reaction may be transformed into —O—NH₂, an aldehyde or a ketone;
in the presence of transglutaminase to form a transaminated peptide of formula [III]

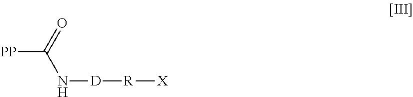

optionally, if X is a latent group, transforming said latent group into —O—NH₂, an aldehyde or a ketone,
said transaminated peptide being further reacted with a second compound of formula [IV]

Y—Z [IV]

wherein Y,
if X represents an aldehyde, a ketone, or a latent group which upon further reaction may be transformed an aldehyde or a ketone, represents —O—NH₂; or,
if X represents —O—NH₂, or a latent group which upon further reaction may be transformed into —O—NH₂, represents an aldehyde or a ketone; and
Z represents the property-modifying group,
to form a derivatized peptide of formula [V]

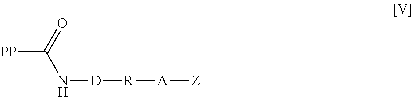

wherein A represents an oxime bond.

Embodiment 47: A pharmaceutical composition according to embodiment 43, wherein D represents —O—.

Embodiment 48: A pharmaceutical composition according to embodiment 43, wherein D represents a single bond.

Embodiment 49: A pharmaceutical composition according to any of embodiments 43 to 45, wherein R represents —(CH₂)₄—CH(NH₂)—CO—NH—CH₂— or —(CH₂)₄—CH(NHCOCH₃)—CO—NH—CH₂—.

Embodiment 50: A pharmaceutical composition according to any of embodiments 43 to 45, wherein R represents C₁₋₆alkylene.

Embodiment 51: A pharmaceutical composition according to embodiment 47, wherein R represents C₁₋₃alkylene.

Embodiment 52: A pharmaceutical composition according to embodiment 48, wherein R represents methylene or propylene.

Embodiment 53: A pharmaceutical composition according to any of embodiments 43 to 49, wherein the compound of formula [II]

H₂N-D-R—X [II]

represents 1,3-diamino-2-propanol, and

Y represents —O—NH₂.

Embodiment 54: A pharmaceutical composition according to any of embodiments 43 to 49, wherein the compound of formula [II]

H₂N-D-R—X [II]

represents 1,3-diaminooxy propane, and

Y represents an aldehyde.

Embodiment 55: A pharmaceutical composition according to 46 to 54, wherein Z is selected amongst

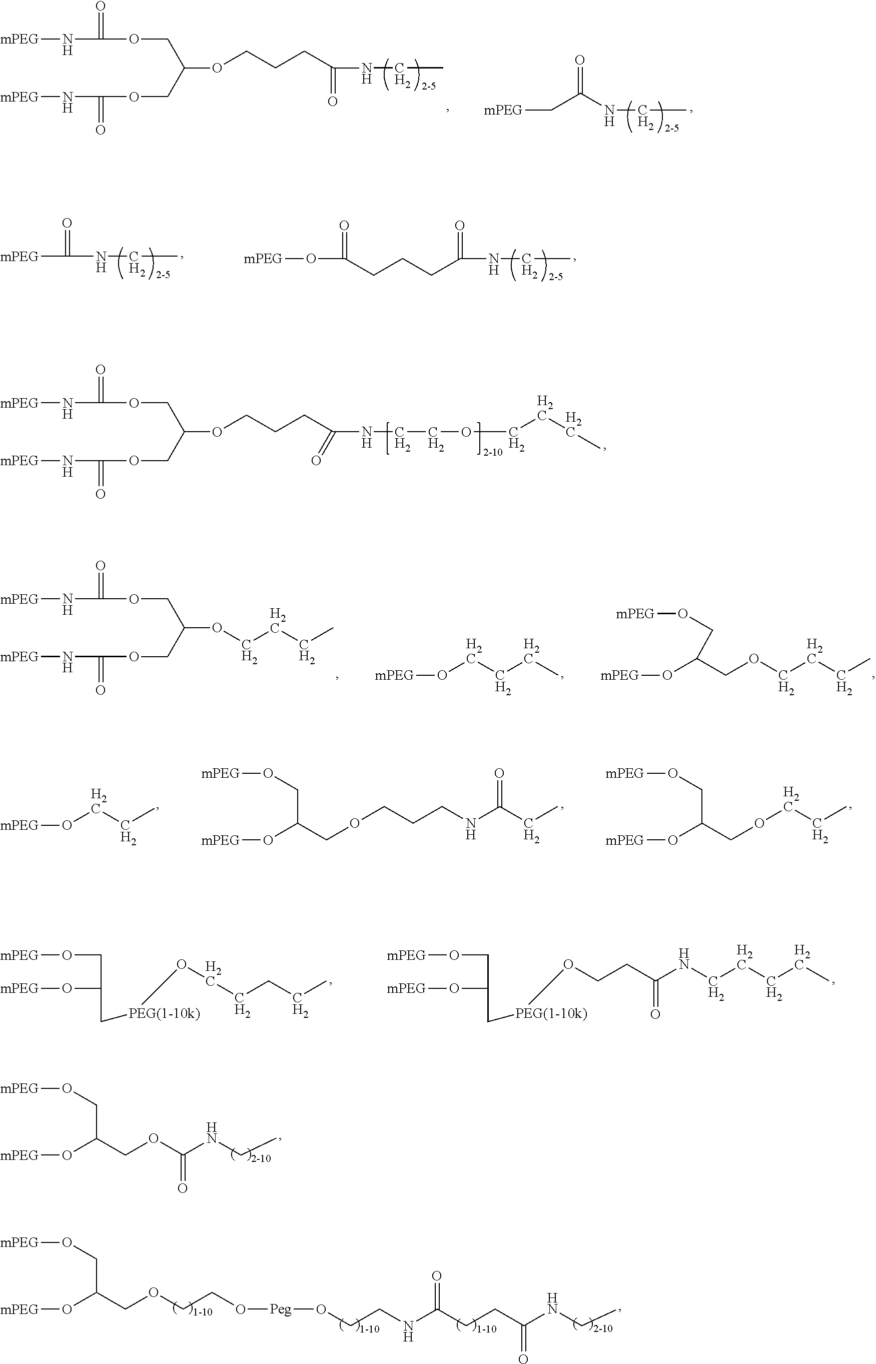

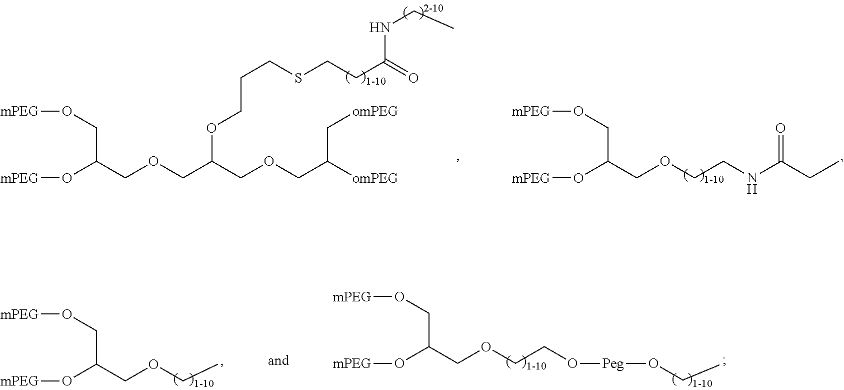

wherein, unless otherwise indicated, mPEG indicates a mPEG with a molecular weight of between 5 kDa and 40 kDa and PEG indicates a PEG with a molecular weight between 1 kDa and 10 kDa;

provided that if Z is

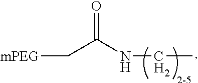

then PEG is 10 kDa PEG.

Embodiment 56: A pharmaceutical composition according to embodiment 55, wherein Z represents

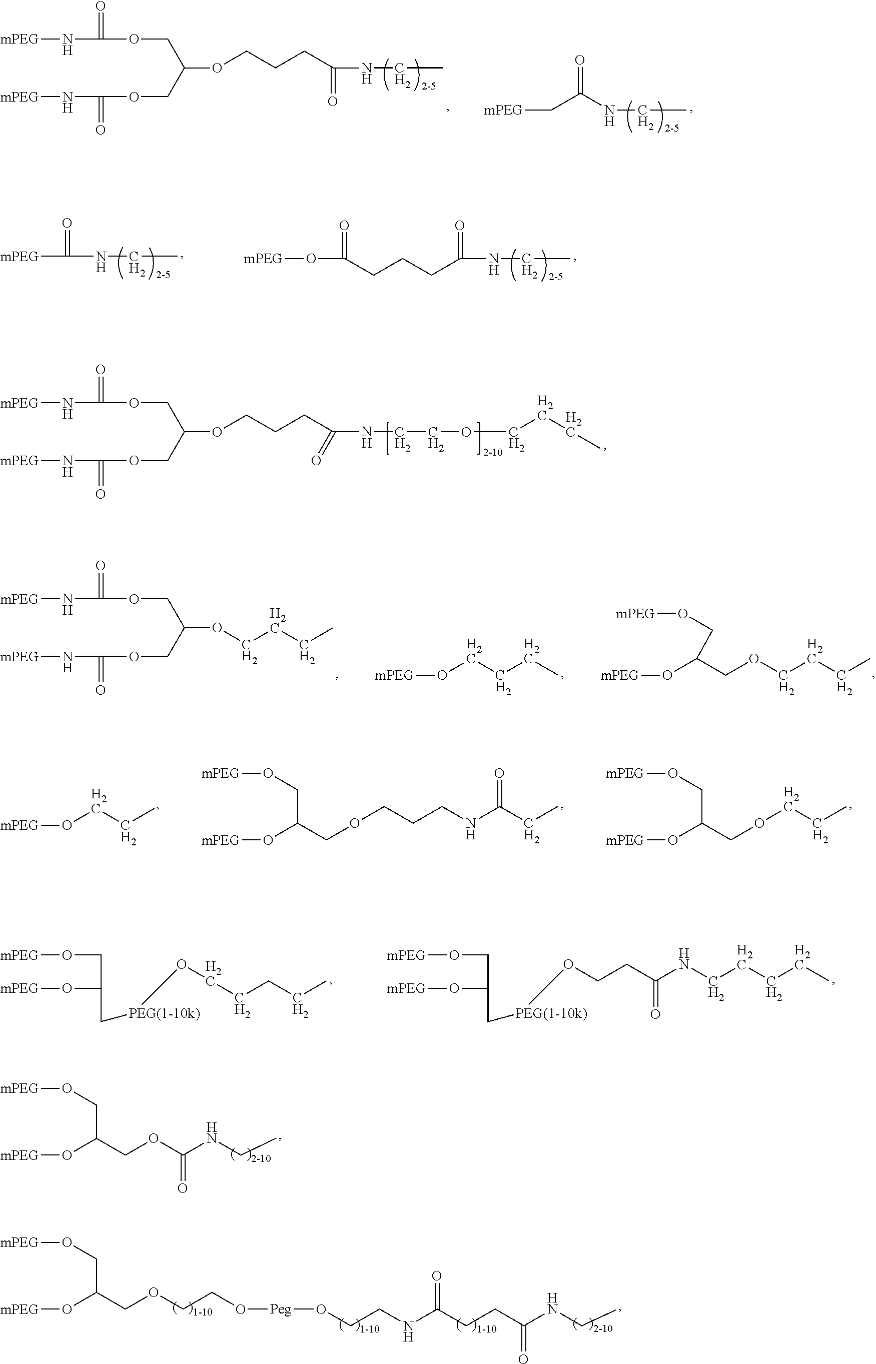

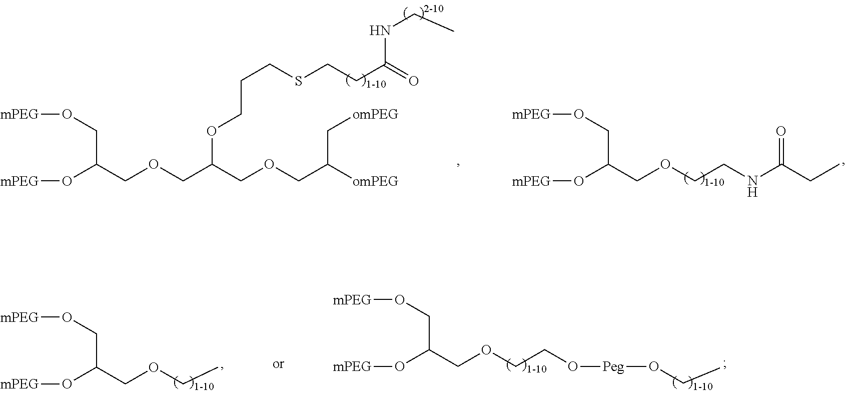

wherein, unless otherwise indicated, mPEG indicates a mPEG with a molecular weight of between 5 kDa and 40 kDa and PEG indicates a PEG with a molecular weight between 1 kDa and 10 kDa;

provided that if Z is

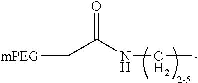

then PEG is 10 kDa PEG.

Embodiment 57: A pharmaceutical composition according to any of embodiments 46 to 56, wherein Y represents —O—NH₂ and X represents an aldehyde or a latent group, which may be further reacted to form an aldehyde.

Embodiment 58: A pharmaceutical composition according to any of embodiments 46 to 56, wherein Y represents —O—NH₂ and X represents a ketone or a latent group which may be further reacted to form a ketone.

Embodiment 59: A pharmaceutical composition according to embodiment 57 or embodiment 58, wherein the compound of formula [IV]

Y—Z [IV]

represents a compound selected from

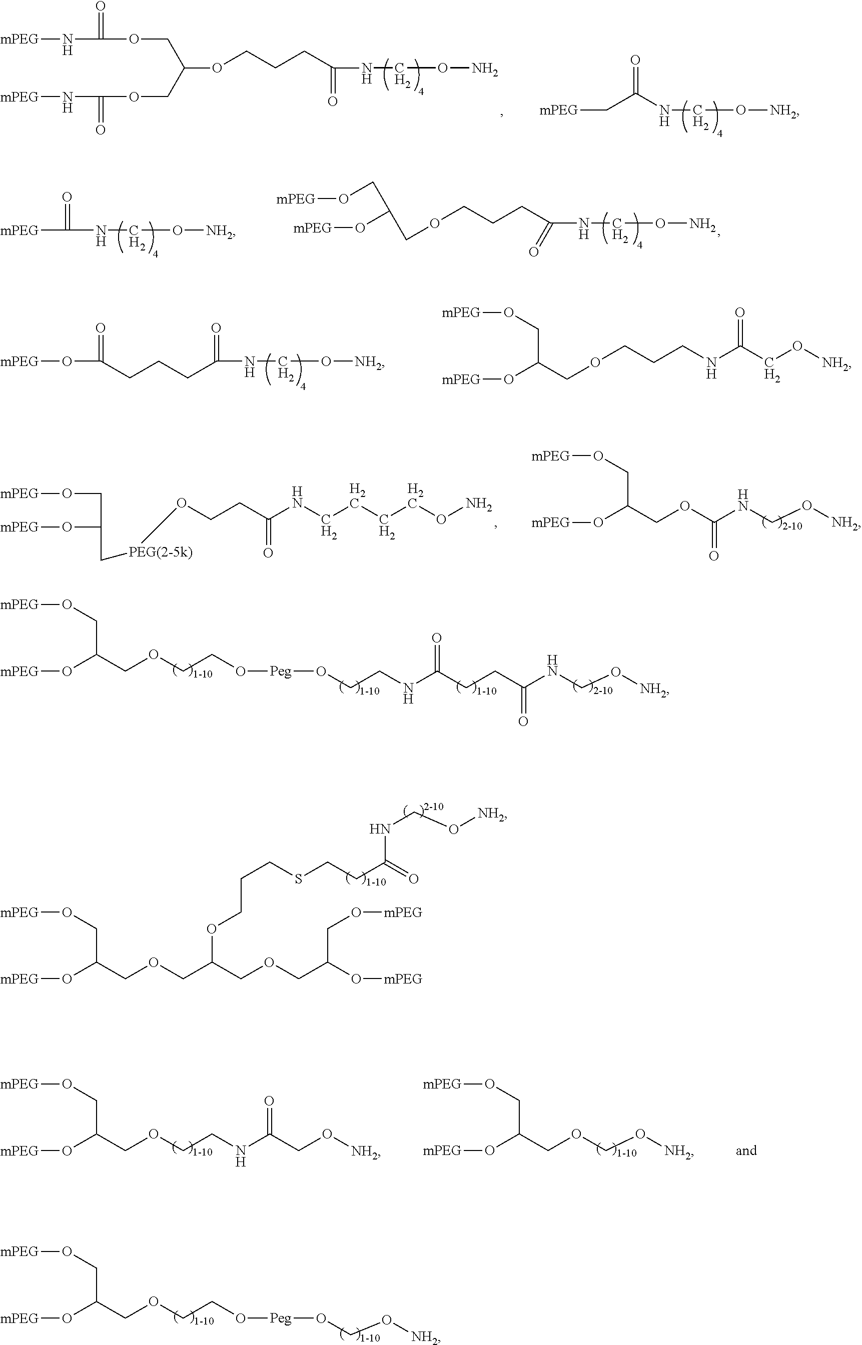

wherein, unless otherwise indicated, mPEG means mPEG with a molecular weight of 10 kDa, 20 kDa or 30 kDa and PEG means PEG with a molecular weight between 2 kDa and 5 kDa.

Embodiment 60: A pharmaceutical composition according to any of embodiments 46 to 56, wherein Y represents an aldehyde and X represent —O—NH₂ or a latent group which upon further reaction may be transformed into —O—NH₂.

Embodiment 61: A pharmaceutical composition according to embodiment 60, wherein the compound of formula [IV]

Y—Z [IV]

represents a compound selected from

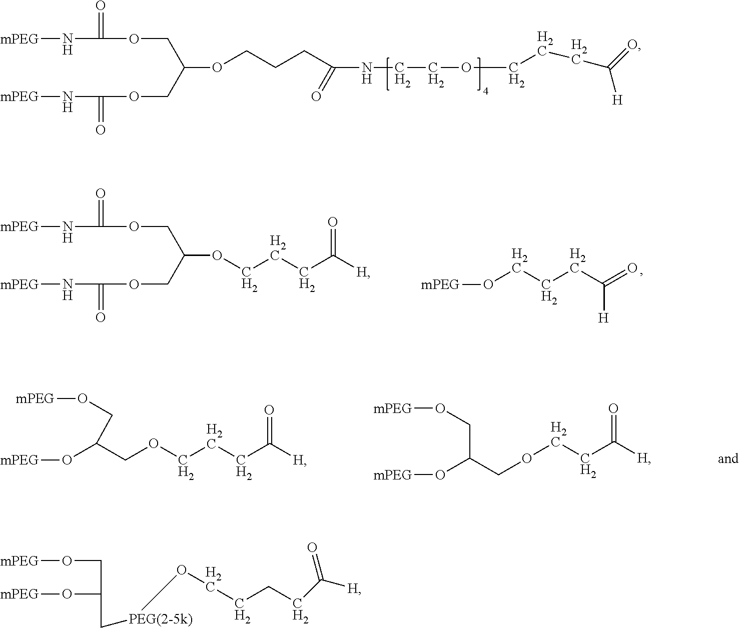

wherein, unless otherwise indicated, mPEG means mPEG with a molecular weight of 10 kDa, 20 kDa or 30 kDa and PEG means PEG with a molecular weight between 2 kDa and 5 kDa.

Embodiment 62: A pharmaceutical composition according to any of embodiments 46 to 56, wherein Y represents a ketone and X represent —O—NH₂ or a latent group which upon further reaction may be transformed into —O—NH₂.

Embodiment 63: A pharmaceutical composition according to embodiment 62, wherein the compound of formula [IV]

Y—Z [IV]

represents a compound selected from

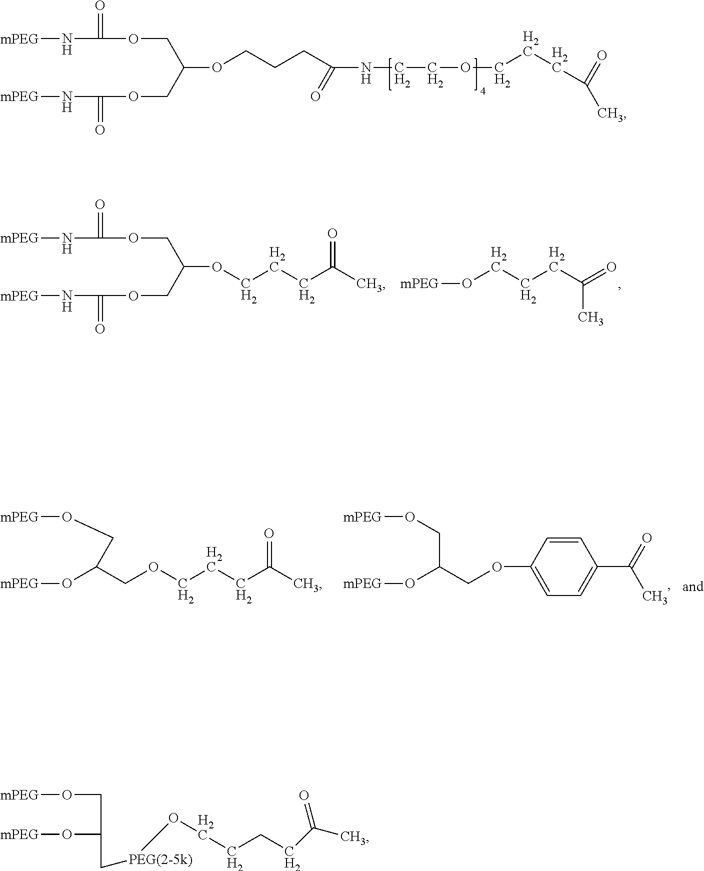

wherein, unless otherwise indicated, mPEG means mPEG with a molecular weight of 10 kDa, 20 kDa or 30 kDa and PEG means PEG with a molecular weight between 2 kDa and 5 kDa.

Embodiment 64: A pharmaceutical composition according to any of embodiments 45 to 63, wherein the therapeutic peptide is a glutamine residue comprising growth hormone compound and PP represents a growth hormone compound radical formally obtained by removing —C(═O)—NH₂ from the side chain of a glutamine residue present in growth hormone compound.

Embodiment 65: A pharmaceutical composition according to embodiment 641, wherein the glutamine residue comprising growth hormone compound exhibits an activity above 20%, such as above 40%, such as above 60%, such as above 80% of the activity of hGH in Assay I.

Embodiment 66: A pharmaceutical composition according to embodiment 64 or embodiment 65, wherein the growth hormone compound has at least 80%, such as at least 85%, such as at least 90%, such as at least 95% such as at least 98% identity with SEQ ID No. 1.

Embodiment 67: A pharmaceutical composition according to any of embodiments 64 to 66, wherein said glutamine residue comprising growth hormone compound is hGH.

Embodiment 68: A pharmaceutical composition according to any of embodiments 64 to 66, wherein said glutamine residue comprising growth hormone compound represents
a) a growth hormone compound comprising the amino acid sequence of SEQ ID No.1,
b) 20 kDa hGH,
c) a growth hormone compound in which the glutamine residue in the position corresponding to position 40 in SEQ ID No. 1 has been deleted or substituted with another amino acid,
d) a growth hormone compound in which the glutamine residue in the position corresponding to position 141 in SEQ ID No. 1 has been deleted or substituted with another amino acid, or
e) a growth hormone compound in which the glutamine residue in the position corresponding to position 40 in SEQ ID No. 1, and the glutamine residue in the position corresponding to position 141 in SEQ ID No. 1 each have been deleted or substituted with another amino acid, and where a glutamine residue is present in another position in the growth hormone compound.

Embodiment 69: A pharmaceutical composition according to any of embodiments 1 to 34, wherein the therapeutic peptide derivatized with a property-modifying group comprises at least one glutamine residue and which derivatized therapeutic peptide is represented by formula [V]

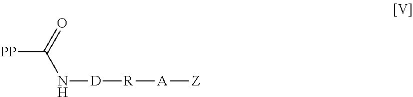

wherein PP represents a radical of the therapeutic peptide formally obtained by removing —C(═O)—NH₂ from the side chain of a glutamine residue present in the peptide;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂— or C₅₋₁₅heteroalkylene;
A represents an oxime bond;

Z represents a property-modifying group.

Embodiment 70: A pharmaceutical composition according to embodiment 69, wherein D represents —O—.

Embodiment 71: A pharmaceutical composition according to embodiment 69, wherein D represents a single bond.

Embodiment 72: A pharmaceutical composition according to any of embodiments 69 to 71, wherein R represents —(CH₂)₄—CH(NH₂)—CO—NH—CH₂— or —(CH₂)₄—CH(NHCOCH₃)—CO—NH—CH₂—.

Embodiment 73. The compound according to any of embodiments 69 to 71, wherein R represents C₁₋₆alkylene.

Embodiment 74: A pharmaceutical composition according to embodiment 730, wherein R represents C₁₋₃alkylene.

Embodiment 75: A pharmaceutical composition according to embodiment 74, wherein R represents methylene or propylene.

Embodiment 76: A pharmaceutical composition according to any of embodiments 69 to 75, wherein Z represents a moiety selected amongst

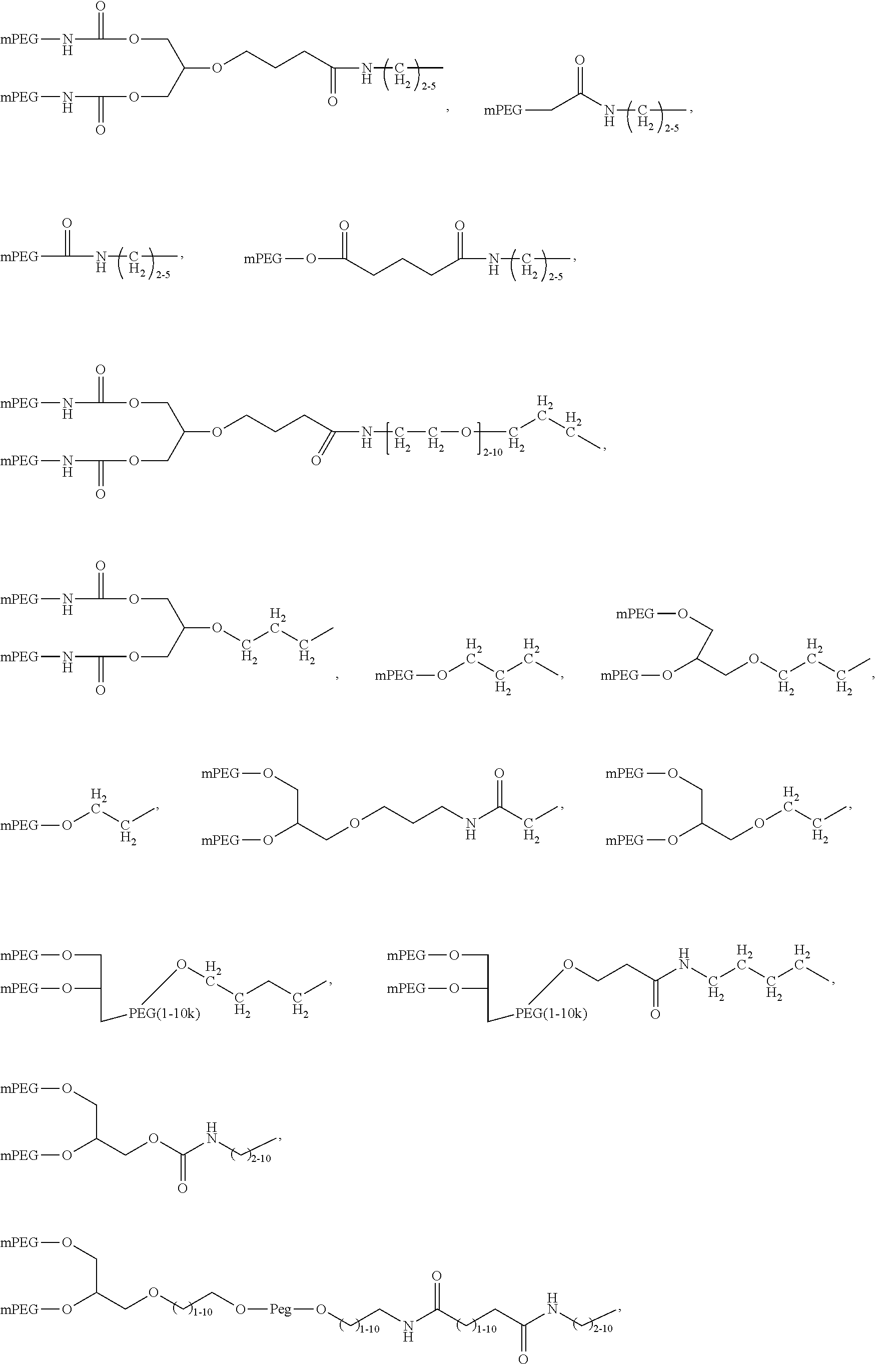

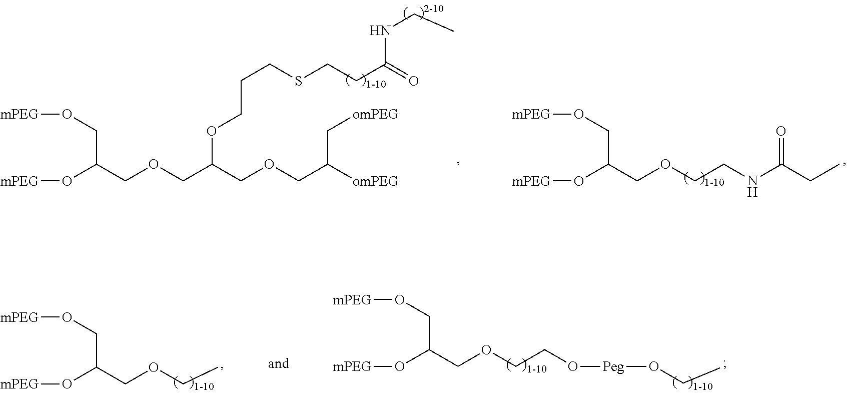

wherein, unless otherwise indicated, mPEG indicates an mPEG with a molecular weight between 5 kDa and 40 kDa and PEG indicates a PEG with a molecular weight between 1 kDa and 10 kDa;

provided that if Z is

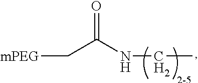

then mPEG is 10 kDa mPEG.

Embodiment 77: A pharmaceutical composition according to embodiment 76, wherein Z represents

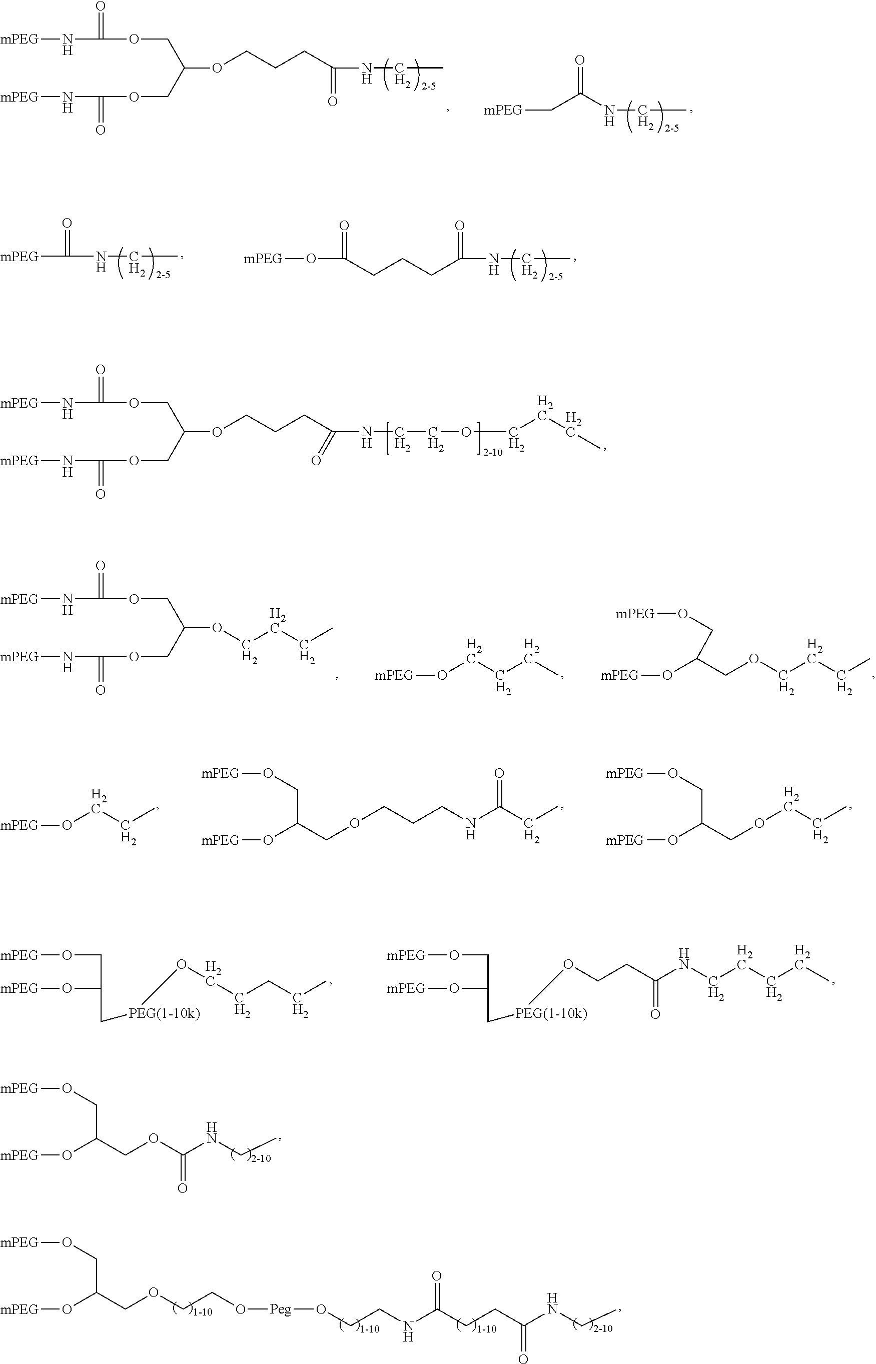

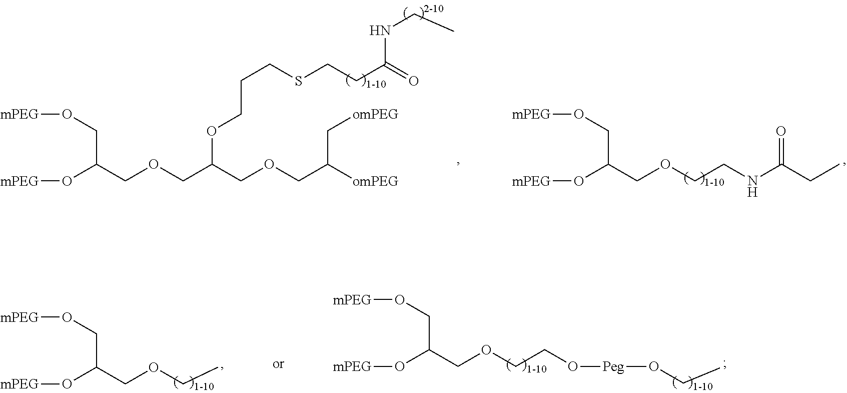

wherein, unless otherwise indicated, mPEG indicates a mPEG with a molecular weight of between 5 kDa and 40 kDa and PEG indicates a PEG with a molecular weight between 1 kDa and 10 kDa;

provided that if Z is

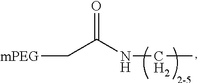

then PEG is 10 kDa PEG.

Embodiment 78: A pharmaceutical composition according to any of embodiments 69 to 77, wherein the therapeutic peptide is a glutamine residue comprising growth hormone compound, and PP represents a growth hormone compound radical formally obtained by removing —C(═O)—NH₂ from the side chain of a glutamine residue present in growth hormone compound.

Embodiment 79: A pharmaceutical composition according to embodiment 78, wherein the glutamine residue comprising growth hormone compound exhibits an activity above 20%, such as above 40%, such as above 60%, such as above 80% of that of hGH in Assay I.

Embodiment 80: A pharmaceutical composition according to embodiment 78 or embodiment 79, wherein the glutamine residue comprising growth hormone compound has at least 80%, such as at least 85%, such as at least 90%, such as at least 95%, such as at least 97%, such as at least 98% or such as at least 99% identity with SEQ ID No. 1.

Embodiment 81: A pharmaceutical composition according to any of Embodiment 78 to 80, wherein the property-modifying group is conjugated to the growth hormone compound in a position corresponding to Gln40 and/or Gln141 in human growth hormone.

Embodiment 82: A pharmaceutical composition according to any of embodiments 78 to 80, wherein said glutamine residue comprising growth hormone compound is hGH.

Embodiment 83: A pharmaceutical composition according to any of embodiments 78 to 80, wherein said glutamine residue comprising growth hormone compound is 20 kDa hGH.

Embodiment 84: A pharmaceutical composition according to any of embodiments 78 to 80, wherein PP represents the radical obtained by removing —C(═O)—NH₂ from the side chain of a glutamine residue present in the glutamine residue comprising growth hormone compound.

Embodiment 85: A pharmaceutical composition according to any of embodiments 78 to 84, wherein PP represents the radical obtained by removing —C(═O)—NH₂ from the side chain of
a) a glutamine residue in the position corresponding to position 40 in SEQ ID No. 1; or
b) a glutamine residue in the position corresponding to position 141 in SEQ ID No. 1;
   or
   PP represents the radical obtained by removing —C(═O)—NH₂ from the side chain of a glutamine residue in the position corresponding to position 40 in SEQ ID No. 1, wherein any glutamine residue in the position corresponding to position 141 in SEQ ID No. 1 has been deleted or substituted with another amino acid;
   or
   PP represents the radical obtained by removing —C(═O)—NH₂ from the side chain of a glutamine residue in the position corresponding to position 141 in SEQ ID No. 1, wherein any glutamine residue in the position corresponding to position 40 in SEQ ID No. 1 has been deleted or substituted with another amino acid;
   or
   PP represents the radical obtained by removing —C(═O)—NH₂ from the side chain of a glutamine residue present in a position different from the position corresponding to position 40 in SEQ ID No. 1 and different from the position corresponding to position 141 in SEQ ID No. 1, wherein any glutamine residue in the position corresponding to position 40 in SEQ ID No. 1, and any glutamine residue in the position corresponding to position 141 in SEQ ID No. 1 each have been deleted or substituted with another amino acid.

Embodiment 86: A pharmaceutical composition according to any of embodiments 1 to 54 or embodiments 69 to 75, wherein the derivatized therapeutic peptide is selected from
N^{δ141/40}-2-(O-(4-(4-(1,3-bis(mPEG(10k)ylaminocarbonyloxy)prop-2-yloxy)butyryl)aminobutyl)-oximino)ethyl hGH;
N^{δ141/40}-2-(O-(4-(4-(mPEG(10k)yloxy)butyryl)aminobutyl)oximino)ethyl hGH;
N^{δ141/40}-2-(O-(4-(3-(mPEG(10k)yloxy)propionyl)aminobutyl)oximino)ethyl hGH;
N^{δ141/40}-2-(O-(4-(4-(2,3-bis(mPEG(10k)yloxy)prop-1-yloxy)butyryl)aminobutyl)oximino)ethyl hGH;
N^{δ141/40}-2-(O-(4-(5-(mPEG(10k)yloxy-5-oxopentanoyl)aminobutyl)oximino)ethyl hGH;
N^{δ141/40}-3-((4-(2-(2-(2-(2-(4-(1,3-bis(mPEG(10k)ylaminocarbonyloxy)prop-2-yloxy)butyryl-amino)ethoxy)ethoxy)ethoxy)ethoxy)butylidene)aminoxy)prop-1-yloxy hGH;
N^{δ141/40}-3-((4-(1,3-bis(mPEG(10k)ylaminocarbonyloxy)prop-2-yloxy)butylidene)-aminoxy)-propyloxy hGH;
N^{δ141/40}-3-((4-(mPEG(10k)yloxy)butylidene)aminoxy)propyloxy hGH;
N^{δ141/40}-3-((4-(2,3-bis(mPEG(10k)yloxy)prop-1-yloxy)butylidene)aminoxy)propyloxy hGH;
N^{δ141/40}-3-((3-(mPEG(10k)yloxy)propylidene)aminoxy)propyloxy hGH;
N^{δ141/40}-2-(O-(2-(3-(2,3-bis(mPEG(10k)yloxy)propyloxy)propylamino)-2-oxoethyl)oximino)ethyl hGH;
N^{δ141/40}-3-((4-(2,3-bis(mPEG(10k)yloxy)prop-1-yloxy)propylidene)aminoxy)propyloxy hGH;
N^{δ141/40}-2-(O-(4-(4-(1,3-bis(mPEG(20k)ylaminocarbonyloxy)prop-2-yloxy)butyryl)aminobutyl)-oximino)ethyl hGH;
N^{δ141/40}-2-(O-(4-(3-(mPEG(20k)yloxy)propionyl)aminobutyl)oximino)ethyl hGH;
N^{δ141/40}-2-(O-(4-(4-(2,3-bis(mPEG(20k)yloxy)prop-1-yloxy)butyryl)aminobutyl)oximino)ethyl hGH;
N^{δ141/40}-2-(O-(4-(5-(mPEG(20k)yloxy-5-oxopentanoyl)aminobutyl)oximino)ethyl hGH;
N^{δ141/40}-3-((4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene)aminoxy)prop-1-yloxy hGH;
N^{δ141/40}-3-((4-(1,3-bis(mPEG(20k)ylaminocarbonyloxy)prop-2-yloxy)butylidene)-aminoxy)-propyloxy hGH;
N^{δ141/40}-3-((4-(mPEG(20k)yloxy)butylidene)aminoxy)propyloxy hGH;
N^{δ141/40}-3-((4-(2,3-bis(mPEG(20k)yloxy)prop-1-yloxy)butylidene)aminoxy)propyloxy hGH;
N^{δ141/40}-3-((3-(mPEG(20k)yloxy)propylidene)aminoxy)propyloxy hGH;
N^{δ141/40}-2-(O-(2-(3-(2,3-bis(mPEG(20k)yloxy)propyloxy)propylamino)-2-oxoethyl)oximino)ethyl hGH;
N^{δ141/40}-3-((4-(2,3-bis(mPEG(20k)yloxy)prop-1-yloxy)propylidene)aminoxy)propyloxy hGH;
N^{δ141/40}-2-(O-(4-(4-(1,3-bis(mPEG(30k)ylaminocarbonyloxy)prop-2-yloxy)butyryl)aminobutyl)-oximino)ethyl hGH;
N^{δ14/40}-2-(O-(4-(3-(mPEG(30k)yloxy)propionyl)aminobutyl)oximino)ethyl hGH;
N^{δ141/40}-2-(O-(4-(4-(2,3-bis(mPEG(30k)yloxy)prop-1-yloxy)butyryl)aminobutyl)oximino)ethyl hGH;
N^{δ141/40}-2-(O-(4-(5-(mPEG(30k)yloxy-5-oxopentanoyl)aminobutyl)oximino)ethyl hGH;
N^{δ141/40}-3-((4-(2-(2-(2-(2-(4-(1,3-bis(mPEG(30k)ylaminocarbonyloxy)prop-2-yloxy)butyryl-amino)ethoxy)ethoxy)ethoxy)ethoxy)butylidene)aminoxy)prop-1-yloxy hGH;
N^{δ141/40}-3-((4-(1,3-bis(mPEG(30k)ylaminocarbonyloxy)prop-2-yloxy)butylidene)-aminoxy)-propyloxy hGH;
N^{δ141/40}-3-((4-(mPEG(30k)yloxy)butylidene)aminoxy)propyloxy hGH;
N^{δ141/40}-3-((4-(2,3-bis(mPEG(30k)yloxy)prop-1-yloxy)butylidene)aminoxy)propyloxy hGH;
N^{δ141/40}-3-((3-(mPEG(30k)yloxy)propylidene)aminoxy)propyloxy hGH;
N^{δ141/40}-2-(O-(2-(3-(2,3-bis(mPEG(30k)yloxy)propyloxy)propylamino)-2-oxoethyl)oximino)ethyl hGH;
N^{δ141/40}-3-((4-(2,3-bis(mPEG(30k)yloxy)prop-1-yloxy)propylidene)aminoxy)propyloxy hGH;
N^{δ141/40}-3-((4-((2,3-bis(mPEG(20k)yl)prop-1-yloxy)PEGyloxy)butylidene)aminoxy)propyloxy hGH;
N^{δ141/40}-2-((4-(4-((2,3-bis(mPEG(20k)yl)propyl)PEGyloxy)butyrylamino)butyl)oximino)ethyl hGH;
N^{δ141}-2-(O-(4-(4-(1,3-bis(mPEG(10k)ylaminocarbonyloxy)prop-2-yloxy)butyryl)-aminobutyl)-oximino)ethyl hGH;
N^{δ141}-2-(O-(4-(4-(mPEG(10k)yloxy)butyryl)aminobutyl)oximino)ethyl hGH;
N^{δ141}-2-(O-(4-(3-(mPEG(10k)yloxy)propionyl)aminobutyl)oximino)ethyl hGH;
N^{δ141}-2-(O-(4-(4-(2,3-bis(mPEG(10k)yloxy)prop-1-yloxy)butyryl)aminobutyl)oximino)ethyl hGH;
N^{δ141}-2-(O-(4-(5-(mPEG(10k)yloxy-5-oxopentanoyl)aminobutyl)oximino)ethyl hGH;
N^{δ141}-3-((4-(2-(2-(2-(2-(4-(1,3-bis(mPEG(10k)ylaminocarbonyloxy)prop-2-yloxy)butyrylamino)-ethoxy)ethoxy)ethoxy)ethoxy)butylidene)aminoxy)prop-1-yloxy hGH;
N^{δ141}-3-((4-(1,3-bis(mPEG(10k)ylaminocarbonyloxy)prop-2-yloxy)butylidene)-aminoxy)-propyloxy hGH;
N^{δ141}-3-((4-(mPEG(10k)yloxy)butylidene)aminoxy)propyloxy hGH;
N^{δ141}-3-((4-(2,3-bis(mPEG(10k)yloxy)prop-1-yloxy)butylidene)aminoxy)propyloxy hGH;
N^{δ141}-3-((3-(mPEG(10k)yloxy)propylidene)aminoxy)propyloxy hGH;
N^{δ141}-2-(O-(2-(3-(2,3-bis(m PEG(10k)yloxy)propyloxy)propylamino)-2-oxoethyl)oximino)ethyl hGH;
N^{δ141}-3-((4-(2,3-bis(mPEG(10k)yloxy)prop-1-yloxy)propylidene)aminoxy)propyloxy hGH;
N^{δ141}-2-(O-(4-(4-(1,3-bis(mPEG(20k)ylaminocarbonyloxy)prop-2-yloxy)butyryl)-minobutyl)-oximino)ethyl hGH;
N^{δ141}-2-(O-(4-(3-(mPEG(20k)yloxy)propionyl)aminobutyl)oximino)ethyl hGH;
N^{δ141}-2-(O-(4-(4-(2,3-bis(mPEG(20k)yloxy)prop-1-yloxy)butyryl)aminobutyl)oximino)ethyl hGH;
N^{δ141}-2-(O-(4-(5-(mPEG(20k)yloxy-5-oxopentanoyl)aminobutyl)oximino)ethyl hGH;
N^{δ141}-3-((4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene)aminoxy)prop-1-yloxy hGH;
N^{δ141}-3-((4-(1,3-bis(mPEG(20k)ylaminocarbonyloxy)prop-2-yloxy)butylidene)-aminoxy)-propyloxy hGH;
N^{δ141}-3-((4-(mPEG(20k)yloxy)butylidene)aminoxy)propyloxy hGH;
N^{δ141}-3-((4-(2,3-bis(mPEG(20k)yloxy)prop-1-yloxy)butylidene)aminoxy)propyloxy hGH;
N^{δ141}-3-((3-(mPEG(20k)yloxy)propylidene)aminoxy)propyloxy hGH;
N^{δ141}-2-(O-(2-(3-(2,3-bis(mPEG(20k)yloxy)propyloxy)propylamino)-2-oxoethyl)oximino)ethyl hGH;
N^{δ141}-3-((4-(2,3-bis(mPEG(20k)yloxy)prop-1-yloxy)propylidene)aminoxy)propyloxy hGH;
N^{δ141}-2-(O-(4-(4-(1,3-bis(mPEG(30k)ylaminocarbonyloxy)prop-2-yloxy)butyryl)-aminobutyl)oximino)ethyl hGH;
N^{δ141}-2-(O-(4-(3-(mPEG(30k)yloxy)propionyl)aminobutyl)oximino)ethyl hGH;
N^{δ141}-2-(O-(4-(4-(2,3-bis(mPEG(30k)yloxy)prop-1-yloxy)butyryl)aminobutyl)oximino)ethyl hGH,
N^{δ141}-2-(O-(4-(5-(mPEG(30k)yloxy-5-oxopentanoyl)aminobutyl)oximino)ethyl hGH;
N^{δ141}-3-((4-(2-(2-(2-(2-(4-(1,3-bis(mPEG(30k)ylaminocarbonyloxy)prop-2-yloxy)butyrylamino)-ethoxy)ethoxy)ethoxy)ethoxy)butylidene)aminoxy)prop-1-yloxy hGH;
N^{δ141}-3-((4-(1,3-bis(mPEG(30k)ylaminocarbonyloxy)prop-2-yloxy)butylidene)-aminoxy)-propyloxy hGH;
N^{δ141}-3-((4-(mPEG(30k)yloxy)butylidene)aminoxy)propyloxy hGH;
N^{δ141}-3-((4-(2,3-bis(mPEG(30k)yloxy)prop-1-yloxy)butylidene)aminoxy)propyloxy hGH;
N^{δ141}-3-((3-(mPEG(30k)yloxy)propylidene)aminoxy)propyloxy hGH;
N^{δ141}-2-(O-(2-(3-(2,3-bis(mPEG(30k)yloxy)propyloxy)propylamino)-2-oxoethyl)oximino)ethyl hGH;
N^{δ141}-3-((4-(2,3-bis(mPEG(30k)yloxy)prop-1-yloxy)propylidene)aminoxy)propyloxy hGH;
N^{δ141}-3-((4-((2,3-bis(mPEG(20k)yloxy)prop-1-yl)PEGyloxy)butylidene)aminoxy)propyloxy hGH;
N^{δ141}-2-((4-(4-((2,3-bis(mPEG(20k)yl)propyl)PEGyloxy)butyrylamino)butyl)oximino)ethyl hGH;
N^{δ40}-2-(O-(4-(4-(1,3-bis(mPEG(10k)ylaminocarbonyloxy)prop-2-yloxy)butyryl)-aminobutyl)-oximino)ethyl hGH;
N^{δ40}-2-(O-(4-(4-(mPEG(10k)yloxy)butyryl)aminobutyl)oximino)ethyl hGH;
N^{δ40}-2-(O-(4-(3-(mPEG(10k)yloxy)propionyl)aminobutyl)oximino)ethyl hGH;
N^{δ40}-2-(O-(4-(4-(2,3-bis(mPEG(10k)yloxy)prop-1-yloxy)butyryl)aminobutyl)oximino)ethyl hGH;
N^{δ40}-2-(O-(4-(5-(mPEG(10k)yloxy-5-oxopentanoyl)aminobutyl)oximino)ethyl hGH;
N^{δ40}-3-((4-(2-(2-(2-(2-(4-(1,3-bis(mPEG(10k)ylaminocarbonyloxy)prop-2-yloxy)butyrylamino)-ethoxy)ethoxy)ethoxy)ethoxy)butylidene)aminoxy)prop-1-yloxy hGH;
N^{δ40}-3-((4-(1,3-bis(mPEG(10k)ylaminocarbonyloxy)prop-2-yloxy)butylidene)-aminoxy)-propyloxy hGH;
N^{δ40}-3-((4-(mPEG(10k)yloxy)butylidene)aminoxy)propyloxy hGH;
N^{δ40}-3-((4-(2,3-bis(mPEG(10k)yloxy)prop-1-yloxy)butylidene)aminoxy)propyloxy hGH;
N^{δ40}-3-((3-(mPEG(10k)yloxy)propylidene)aminoxy)propyloxy hGH;
N^{δ40}-2-(O-(2-(3-(2,3-bis(mPEG(10k)yloxy)propyloxy)propylamino)-2-oxoethyl)oximino)ethyl hGH;
N^{δ40}-3-((4-(2,3-bis(mPEG(10k)yloxy)prop-1-yloxy)propylidene)aminoxy)propyloxy hGH;
N^{δ40}-2-(O-(4-(4-(1,3-bis(mPEG(20k)ylaminocarbonyloxy)prop-2-yloxy)butyryl)-aminobutyl)oximino)ethyl hGH;
N^{δ40}-2-(O-(4-(3-(mPEG(20k)yloxy)propionyl)aminobutyl)oximino)ethyl hGH;
N^{δ40}-2-(O-(4-(4-(2,3-bis(mPEG(20k)yloxy)prop-1-yloxy)butyryl)aminobutyl)oximino)ethyl hGH;
N^{δ40}-2-(O-(4-(5-(mPEG(20k)yloxy-5-oxopentanoyl)aminobutyl)oximino)ethyl hGH;
N^{δ40}-3-((4-(2-(2-(2-(2-(4-(1,3-bis(mPEG(20k)ylaminocarbonyloxy)prop-2-yloxy)butyrylamino)-ethoxy)ethoxy)ethoxy)ethoxy)butylidene)aminoxy)prop-1-yloxy hGH;
N^{δ40}-3-((4-(1,3-bis(mPEG(20k)ylaminocarbonyloxy)prop-2-yloxy)butylidene)-aminoxy)-propyloxy hGH;
N^{δ40}-3-((4-(mPEG(20k)yloxy)butylidene)aminoxy)propyloxy hGH;
N^{δ40}-3-((4-(2,3-bis(mPEG(20k)yloxy)prop-1-yloxy)butylidene)aminoxy)propyloxy hGH;
N^{δ40}-3-((3-(mPEG(20k)yloxy)propylidene)aminoxy)propyloxy hGH;
N^{δ40}-2-(O-(2-(3-(2,3-bis(mPEG(20k)yloxy)propyloxy)propylamino)-2-oxoethyl)oximino)ethyl hGH,
N^{δ40}-3-((4-(2,3-bis(mPEG(20k)yloxy)prop-1-yloxy)propylidene)aminoxy)propyloxy hGH;
N^{δ40}-2-(O-(4-(4-(1,3-bis(mPEG(30k)ylaminocarbonyloxy)prop-2-yloxy)butyryl)-aminobutyl)-oximino)ethyl hGH;
N^{δ40}-2-(O-(4-(3-(mPEG(30k)yloxy)propionyl)aminobutyl)oximino)ethyl hGH;
N^{δ40}-2-(O-(4-(4-(2,3-bis(mPEG(30k)yloxy)prop-1-yloxy)butyryl)aminobutyl)oximino)ethyl hGH;
N^{δ40}-2-(O-(4-(5-(mPEG(30k)yloxy-5-oxopentanoyl)aminobutyl)oximino)ethyl hGH;
N^{δ40}-3-((4-(2-(2-(2-(2-(4-(1,3-bis(mPEG(30k)ylaminocarbonyloxy)prop-2-yloxy)butyrylamino)-ethoxy)ethoxy)ethoxy)ethoxy)butylidene)aminoxy)prop-1-yloxy hGH;
N^{δ40}-3-((4-(1,3-bis(mPEG(30k)ylaminocarbonyloxy)prop-2-yloxy)butylidene)-aminoxy)-propyloxy hGH;
N^{δ40}-3-((4-(mPEG(30k)yloxy)butylidene)aminoxy)propyloxy hGH;
N^{δ40}-3-((4-(2,3-bis(mPEG(30k)yloxy)prop-1-yloxy)butylidene)aminoxy)propyloxy hGH;
N^{δ40}-3-((3-(mPEG(30k)yloxy)propylidene)aminoxy)propyloxy hGH;
N^{δ40}-2-(O-(2-(3-(2,3-bis(mPEG(30k)yloxy)propyloxy)propylamino)-2-oxoethyl)oximino)ethyl hGH;
N^{δ40}-3-((4-(2,3-bis(mPEG(30k)yloxy)prop-1-yloxy)propylidene)aminoxy)propyloxy hGH;
N^{δ40}-3-((4-(2,3-bis(mPEG(20k)yloxy)prop-1-yl)PEGyloxy)butylidene)aminoxy)propyloxy hGH;
N^{δ40}-2-((4-(4-((2,3-bis(mPEG(20k)yl)propyl)PEGyloxy)butyrylamino)butyl)oximino)ethyl hGH;
N^{ε}141-(2-O-(4-(4-(1,3-bis(m PEG(20K)aminocarbonyloxy)-2-propyloxy)butyrylamino)butyl)-oxyimino)ethyl) hGH;
N^{ε}141-(2-(O-(4-(4-(mPEG(30K)oxy)butyrylamino)-butyl)oxyimino)-ethyl) hGH;
N^{ε}141-(3-((4-(2-(2-(2-(2-(4-(1,3-bis(mPEG(20K)ylaminocarbonyloxy)-2-propyloxy)butyryl-amino)ethoxy)ethoxy)ethoxy)ethoxy)butylidene)aminoxy)propyloxy) hGH;
N^{ε}141-(2-(2-(2,3-bis(mPEG(20K)yloxy)propyloxycarbonylamino)ethyloximino)ethyl) hGH;
N^{ε}141-(2-(O-(2-(2-(mPEG(40K)yloxy)ethylamino)-2-oxoethyl)oximino)ethyl) hGH;
N^{ε}141-(2-(3-(4-((1,3-bis(mPEG(30K)ylaminocarbonyloxy)-2-propyloxy)butylidene)aminoxy)-propyloxyimino)ethyl) hGH;
N^{ε}141/40-(2-O-(4-(4-(1,3-bis(mPEG(20K)aminocarbonyloxy)-2-propyloxy)butyrylamino)-butyl)oxyimino)ethyl) hGH;
N^{ε}141/40-(2-(O-(4-(4-(mPEG(30K)oxy)butyrylamino)-butyl)oxyimino)-ethyl) hGH;
N^{ε}141/40-(3-((4-(2-(2-(2-(2-(4-(1,3-bis(mPEG(20K)ylaminocarbonyloxy)-2-propyloxy)butyryl-amino)ethoxy)ethoxy)ethoxy)ethoxy)butylidene)aminoxy)propyloxy) hGH;
N^{ε}141/40-(2-(2-(2,3-(mPEG(20K)yloxy)propyloxycarbonylamino)ethyloximino)ethyl) hGH;
N^{ε}141/40-(2-(O-(2-(2-(mPEG(40K)yloxy)ethylamino)-2-oxoethyl)oximino)ethyl) hGH;
N^{ε}141/40-(2-(3-(4-((1,3-bis(mPEG(30K)ylaminocarbonyloxy)-2-propyloxy)butylidene)-aminoxy)propyloxyimino)ethyl) hGH;
N^{ε}40-(2-O-(4-(4-(1,3-bis(mPEG(20K)aminocarbonyloxy)-2-propyloxy)butyrylamino)butyl)-oxyimino)ethyl) hGH;
N^{ε}40-(2-(O-(4-(4-(mPEG(30K)oxy)butyrylamino)-butyl)oxyimino)-ethyl) hGH;
N^{ε}40-(3-((4-(2-(2-(2-(2-(4-(1,3-bis(mPEG(20K)ylaminocarbonyloxy)-2-propyloxy)butyryl-amino)ethoxy)ethoxy)ethoxy)ethoxy)butylidene)aminoxy)propyloxy) hGH;
N^{ε}40-(2-(2-(2,3-(mPEG(20K)yloxy)propyloxycarbonylamino)ethyloximino)ethyl) hGH;
N^{ε}40-(2-(O-(2-(2-(mPEG(40K)yloxy)ethylamino)-2-oxoethyl)oximino)ethyl) hGH;

N^{ε}40-(2-(3-(4-((1,3-bis(mPEG(30K)ylaminocarbonyloxy)-2-propyloxy)butylidene)aminoxy)-propyloxyimino)ethyl) hGH;

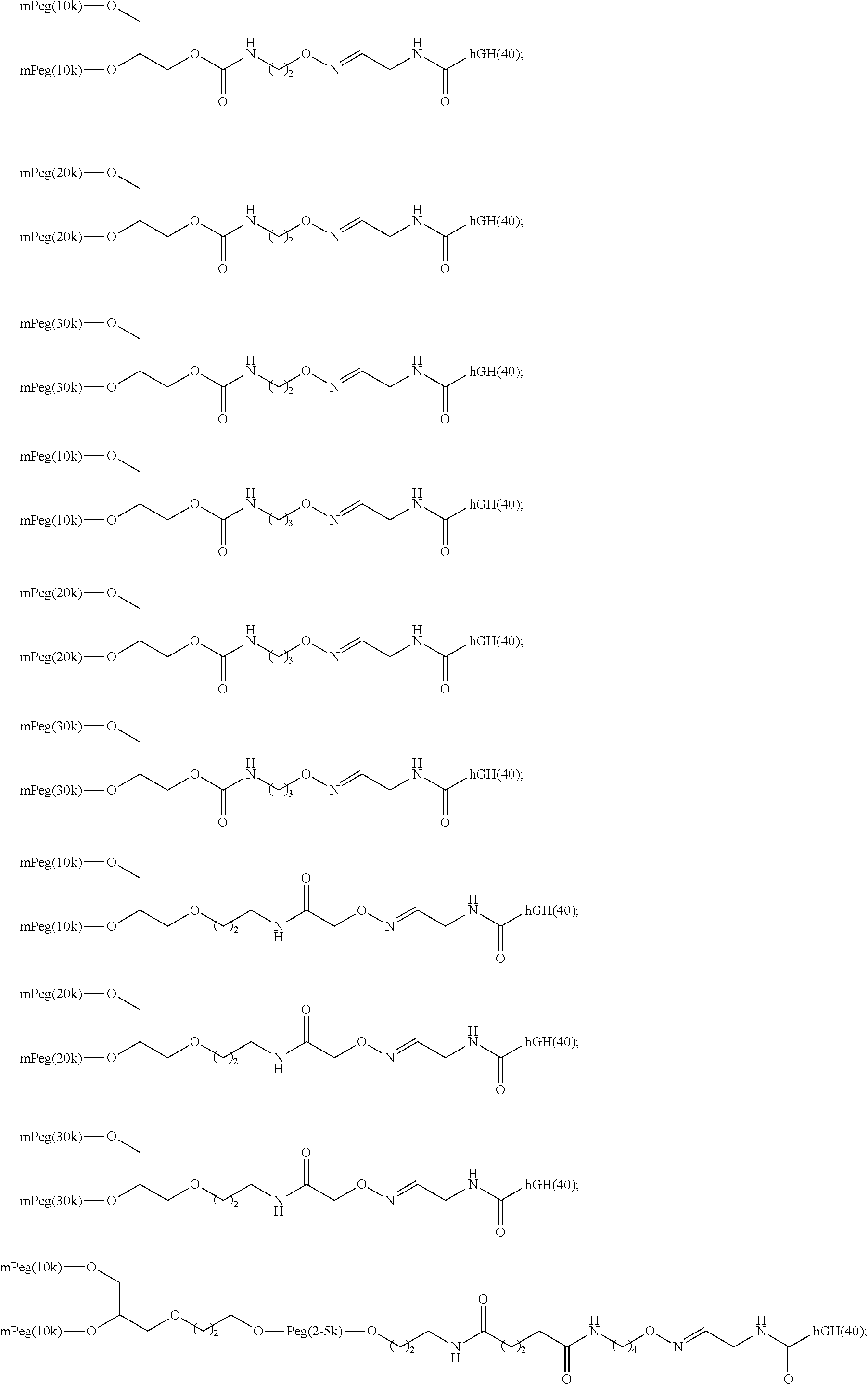

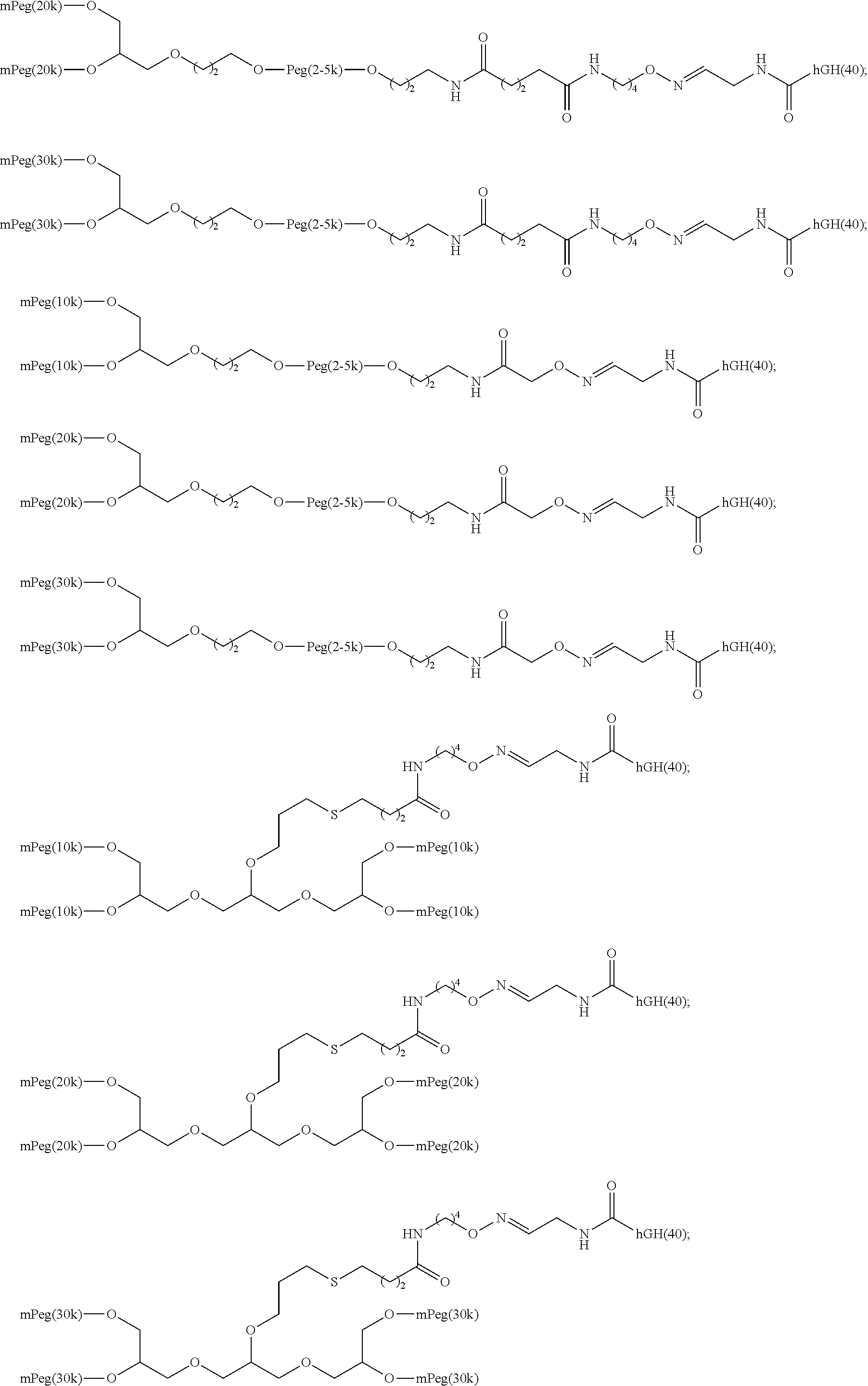

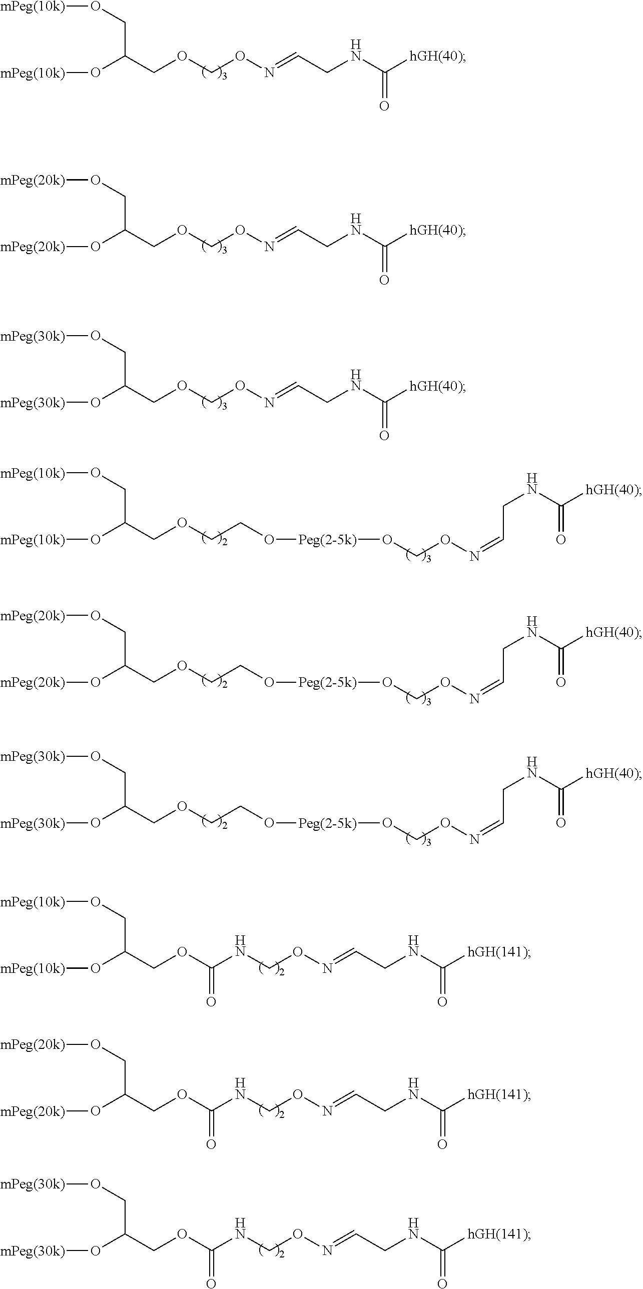

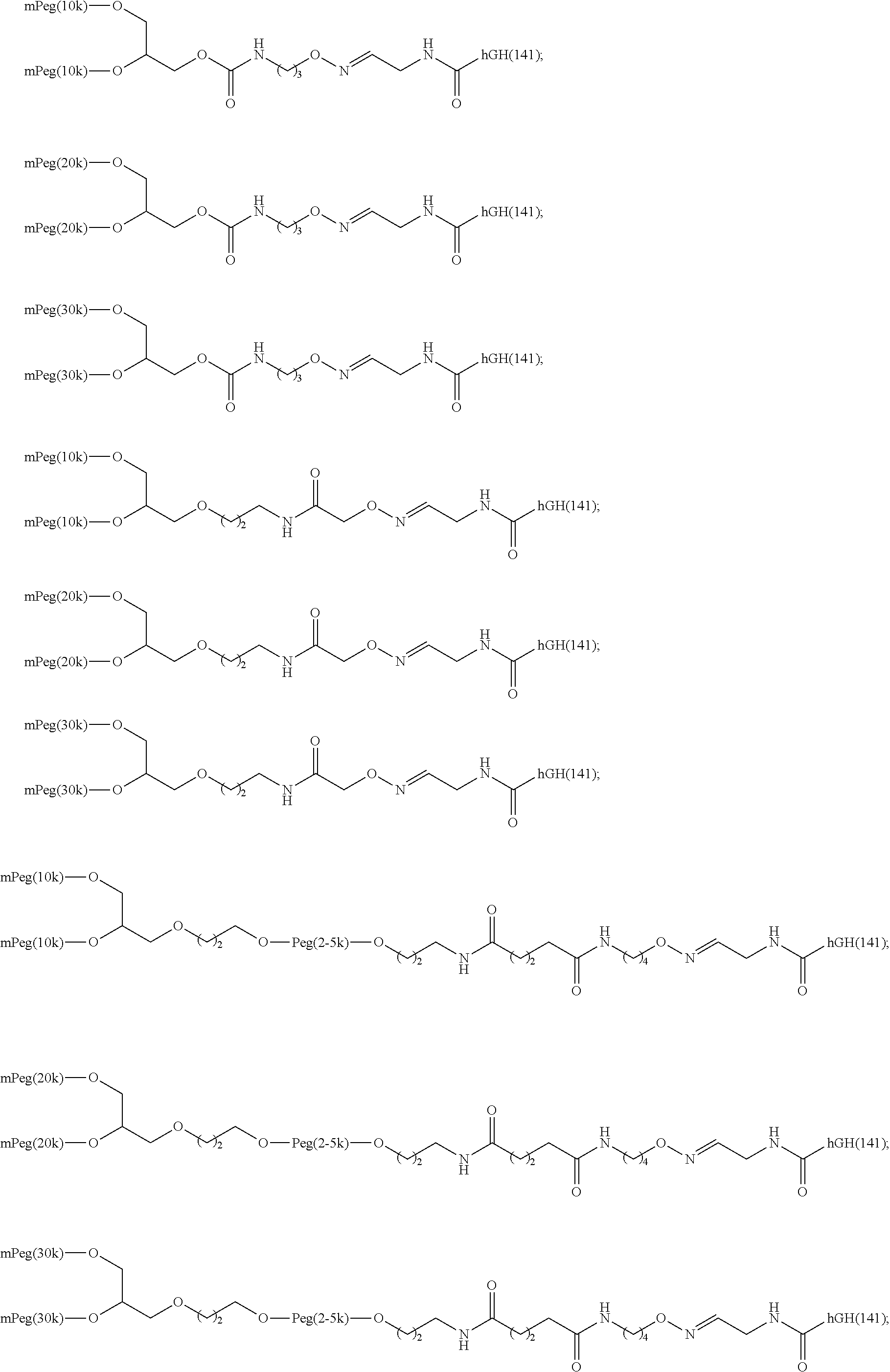

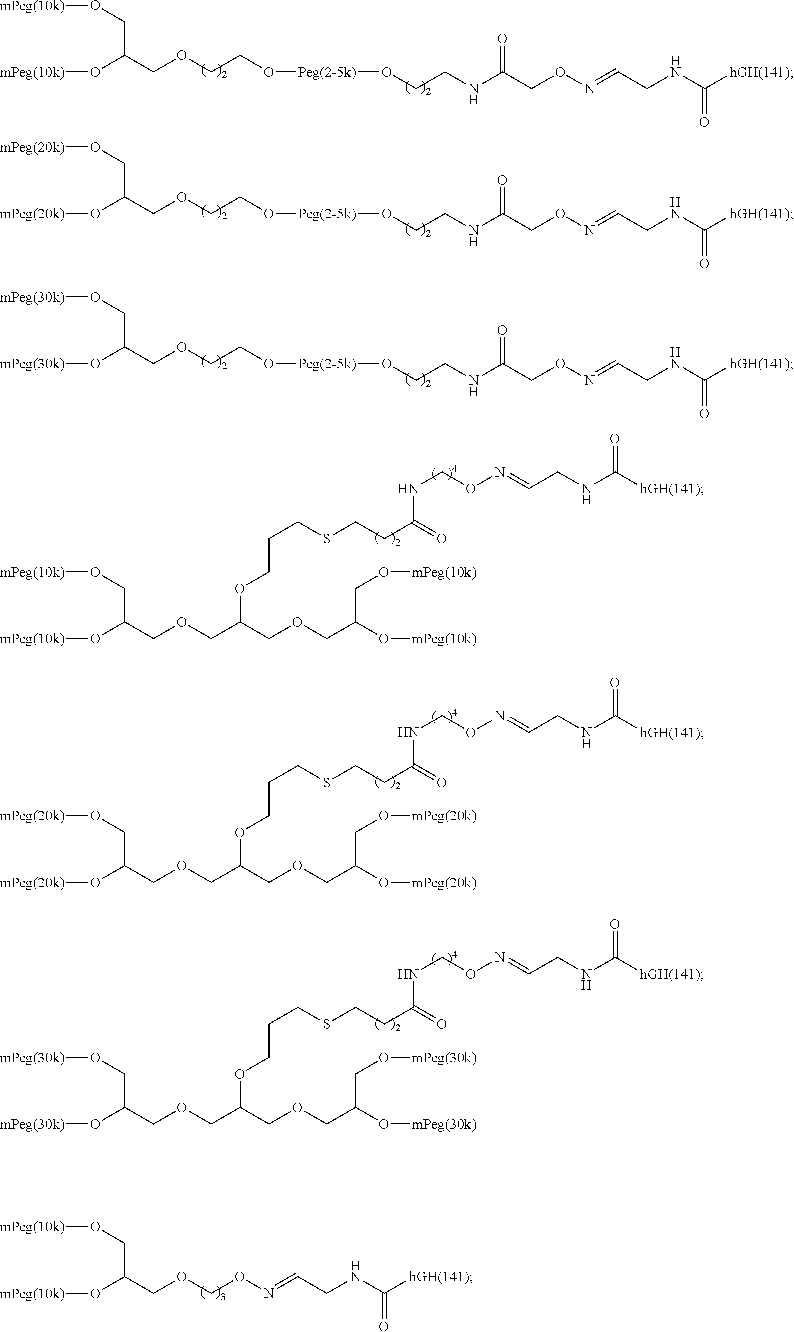

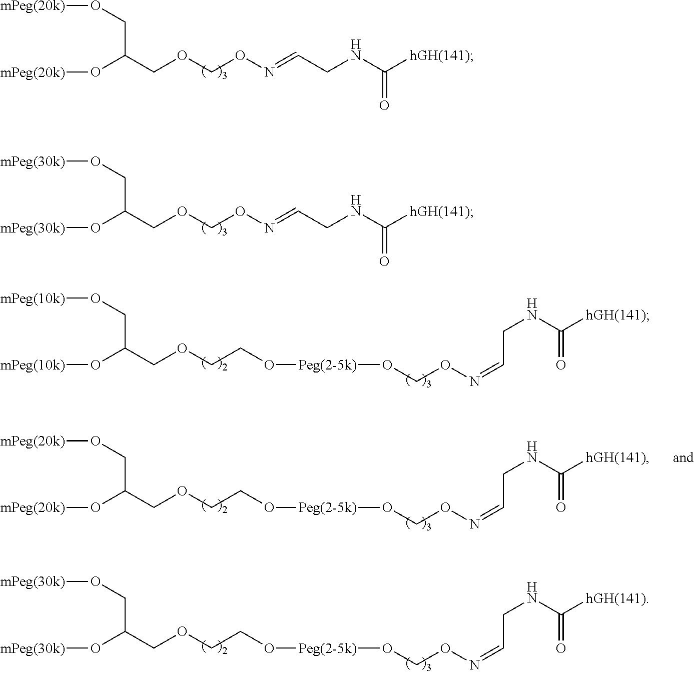

Embodiment 87: A pharmaceutical composition according to any of embodiments 42 to 45, embodiments 64 to 68 or embodiments 78 to 86, wherein the derivatized growth hormone is present in a concentration from 1.0-10 mg (underivatized growth hormone)/ml, such as from 2-8 mg/ml or such as from 3-7 mg/ml.

Embodiment 88: A pharmaceutical composition according to any of embodiments 1 to 87, wherein the peptide derivative is stable for at least 6 months.

Embodiment 89: A pharmaceutical composition according to any of embodiments 1 to 87, wherein the oxime bond is stable for at least 6 months.

Embodiment 89: A pharmaceutical composition according to any of embodiments 1 to 87, wherein the property-modifying group is a PEG or mPEG group and said PEGylated peptide is stable for at least 6 months.

Embodiment 90: A pharmaceutical composition according to embodiments 89, wherein de-PEGylation of said PEGylated peptide is at most 5% during storage at 5° C. for 6 months.

Embodiment 91: A pharmaceutical composition according to embodiments 90, wherein de-PEGylation of said PEGylated peptide is at most 5%, such as at most 4.5% or such as at most 4% during storage at 5° C. for such as at least 6 months, such as 12 months, such as 18 months or such as 24 months.

Embodiment 92: A pharmaceutical composition according to any of the embodiments 88 to 91, where in the derivatized peptides is a growth hormone compound.

Embodiment 93: A pharmaceutical composition according to any of the embodiments 88 to 91, where in the derivatized peptides is a human growth hormone.

Embodiment 94: A pharmaceutical composition according to any of embodiments 1 to 93 for use in therapy.

Embodiment 95: A pharmaceutical composition according to any of embodiments 42 to 45, embodiments 64 to 68, embodiments 79 to 87 or embodiments 92 to 933 for use in therapy.

Embodiment 96: A pharmaceutical composition according to embodiment 95 for use in treatment of diseases benefiting from an increase in the level of circulating growth hormone.

Embodiment 97: A pharmaceutical composition according to embodiment 96, wherein said disease is selected from growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; short stature due to glucocorticoid treatment in children; acceleration of the healing of muscle tissue, nervous tissue or wounds; the acceleration or improvement of blood flow to damaged tissue; or the decrease of infection rate in damaged tissue.

Embodiment 98: A pharmaceutical composition according to any of embodiments 95 to 97, wherein said pharmaceutical composition is to be administered according to a dosing range of from 10 μg/kg/week to 100 μg/kg/week, wherein the amount of the therapeutic peptide is measured as weight of underivatized growth hormone compound.

Embodiment 99: A pharmaceutical composition according to embodiment 98, wherein said pharmaceutical composition is to be administered according to a dosing range of from 10 μg/kg/week to 45 μg/kg/week, wherein the amount of the therapeutic peptide is measured as weight of underivatized growth hormone compound.

Embodiment 100: A pharmaceutical composition according to embodiment 99, wherein said pharmaceutical composition is to be administered according to a dosing range of from 10 μg/kg/week, 15 μg/kg/week, 20 μg/kg/week, 25 μg/kg/week, 30 μg/kg/week, 35 μg/kg/week, 40 μg/kg/week, or 45 μg/kg/week, wherein the amount of the therapeutic peptide is measured as weight of underivatized growth hormone compound.

Embodiment 101: Use of a pharmaceutical composition according to any of embodiments 42 to 45, embodiments 64 to 68, embodiments 78 to 87 or embodiments 92 to 93 for treatment of diseases benefiting from an increase in the level of circulating growth hormone.

Embodiment 102: Use according to embodiment 101, wherein said disease is selected from growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; short stature due to glucocorticoid treatment in children; acceleration of the healing of muscle tissue, nervous tissue or wounds; the acceleration or improvement of blood flow to damaged tissue; or the decrease of infection rate in damaged tissue.

Embodiment 103: Use according to embodiment 101 or embodiment 102, wherein said pharmaceutical composition is to be administered according to a dosing range of from 10 μg/kg/week to 100 μg/kg/week, wherein the amount of the therapeutic peptide is measured as weight of underivatized growth hormone compound.

Embodiment 104: Use according to embodiment 103, wherein said pharmaceutical composition is to be administered according to a dosing range of from 10 μg/kg/week to 45 μg/kg/week, wherein the amount of the therapeutic peptide is measured as weight of underivatized growth hormone compound.

Embodiment 105: Use according to embodiment 104, wherein said pharmaceutical composition is to be administered according to a dosing range of from 10 μg/kg/week, 15 μg/kg/week, 20 μg/kg/week, 25 μg/kg/week, 30 μg/kg/week, 35 μg/kg/week, 40 μg/kg/week, or 45 μg/kg/week, wherein the amount of the therapeutic peptide is measured as weight of underivatized growth hormone compound.

Embodiment 106: Use according to any of the embodiments 94 to 105, wherein the pharmaceutical composition is for administration in the morning, such as within 2 hours before or 2 hours after breakfast.

Embodiment 107: A method of treatment of a disease benefiting from an increase in the level of circulating growth hormone, wherein said method comprising administration of a pharmaceutical composition according to any of embodiments 42 to 45, embodiments 64 to 68, embodiments 78 to 87 or embodiments 92 to 93 to a subject in need thereof.

Embodiment 108: A method according to embodiment 96, wherein said disease is selected from growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; short stature due to glucocorticoid treatment in children; acceleration of the healing of muscle tissue, nervous tissue or wounds; the acceleration or improvement of blood flow to damaged tissue; or the decrease of infection rate in damaged tissue.

Embodiment 109: A method according to embodiment 107 or embodiment 108, wherein said pharmaceutical composition is to be administered according to a dosing range of from 10 μg/kg/week to 100 μg/kg/week, wherein the amount of the therapeutic peptide is measured as weight of underivatized growth hormone compound.

Embodiment 110: A method according to embodiment 109, wherein said pharmaceutical composition is to be administered according to a dosing range of from 10 μg/kg/week to 45 μg/kg/week, wherein the amount of the therapeutic peptide is measured as weight of underivatized growth hormone compound.

Embodiment 111: A method according to embodiment 110, wherein said pharmaceutical composition is to be administered according to a dosing range of from 10 μg/kg/week, 15 μg/kg/week, 20 μg/kg/week, 25 μg/kg/week, 30 μg/kg/week, 35 μg/kg/week, 40 μg/kg/week, or 45 μg/kg/week, wherein the amount of the therapeutic peptide is measured as weight of underivatized growth hormone compound.

Embodiment 112: A method according to any of the embodiments 108 to 111, wherein the pharmaceutical composition to be administered in the morning, such as within 2 hour before or 2 hour after breakfast.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way,

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (for instance all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (“for instance”, “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (for instance a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

### EXAMPLES

### Example 1

### Preparation of N^{ε141}-(2-(2-(2,3-bis(mPeg(20K)yloxy)-propyloxycarbonylamino)-ethyloximino)-ethyl) hGH

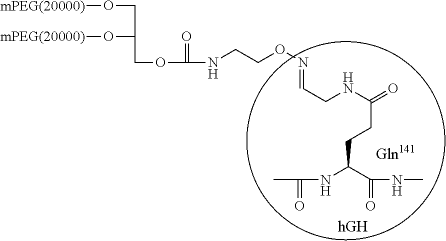

The compound N^{ε141}-(2-(2-(2,3-bis(mPeg(20K)yloxy)-propyloxycarbonylamino)-ethyl-oximino)-ethyl) hGH (“PEG-(Gln-141) hGH”) was prepared as described in WO2006/134148.

### Example 2

### Effects on Choice of pH and Buffer on Stability of Oxime-Linker

SEC-HPLC was performed on a Agilent 1100 series Chromatograph. The SEC samples were analysed with a isocratic mobile phase of 0.063 M Phosphate Buffer, containing 3% isopropanol pH 7.0, on a Phenomenex BioSep 3000 column.

PEG-(Gln-141) hGH was dissolved (3 mg/ml) in different 20 mM buffers at various pH. After incubation at 40° C. for 1 week, the purity of PEG-hGH was determined with SEC-HPLC.

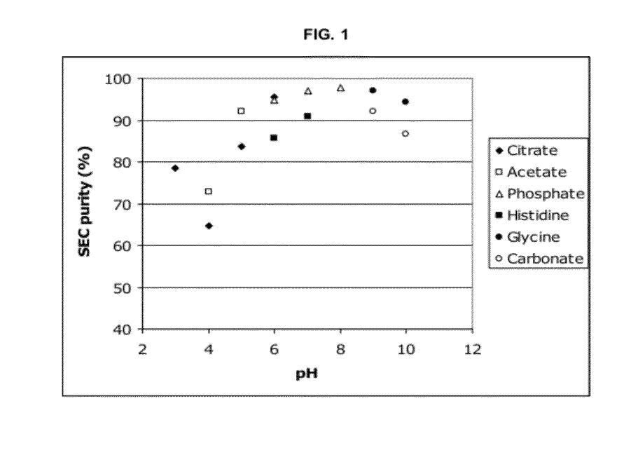

No high molecular species was observed. An impurity peak corresponding to the molecular weight of hGH was found indicating that the PEG moiety was detached. A pH optimum around 7-8 was found to be optimal with relation to the conjugation stability. See FIG. 1. It was also observed that the nature of the buffer species had a high impact on the oxime stability.

To access long-term stability data from some formulations in a short time, a stability study performed at 40° C., 25° C. and 15° C. was performed.

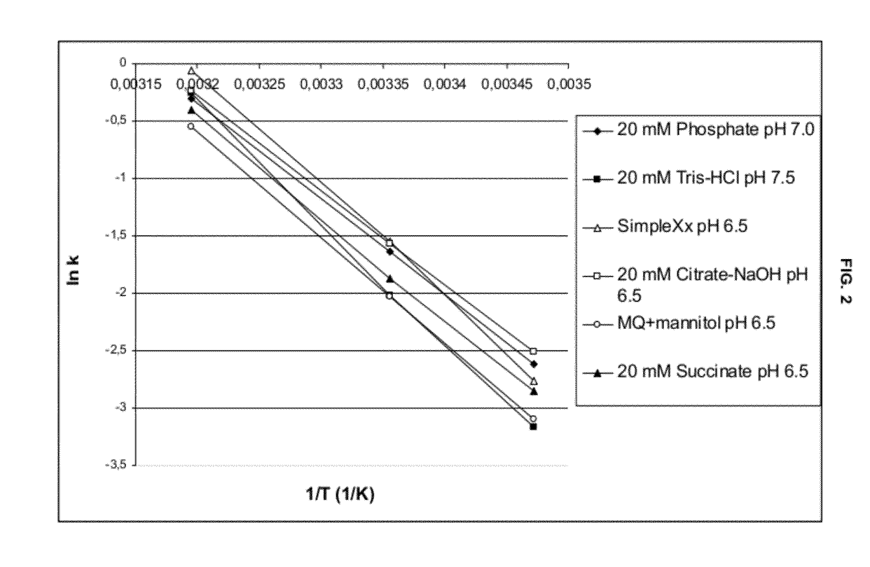

PEG-(Gln-141) hGH was formulated in different 20 mM buffers (+0.5% phenol) at a concentration of 10 mg/ml. Samples were withdrawn after 2, 5 and 7 days in order to obtain the degradation rate constant for the formulations at each temperature, The rate constants are shown in Table 1. These rates were plotted in an Arrhenius plot, which can be seen in FIG. 2.

TABLE 1
Reaction rates (area-%/day) for PEG-linker degradation
at different formulations and temperatures

| | 15° C. | 25° C. | 40° C. |
|---|---|---|---|
| 20 mM Phosphate pH 7.0 | 0.0728 | 0.1937 | 0.7395 |
| 20 mM Tris pH 7.5 | 0.042 | 0.1329 | 0.7741 |
| SimpleXx buffer pH 6.5 | 0.0629 | 0.2123 | 0.9471 |
| 20 mM Citrate-NaOH pH 6.5 | 0.0809 | 0.2082 | 0.7923 |
| MQ-water pH 6.5 + Mannitol 20 mg/ml | 0.045 | 0.1313 | 0.5798 |
| 20 mM Succinate pH 6.5 | 0.0574 | 0.1531 | 0.6691 |

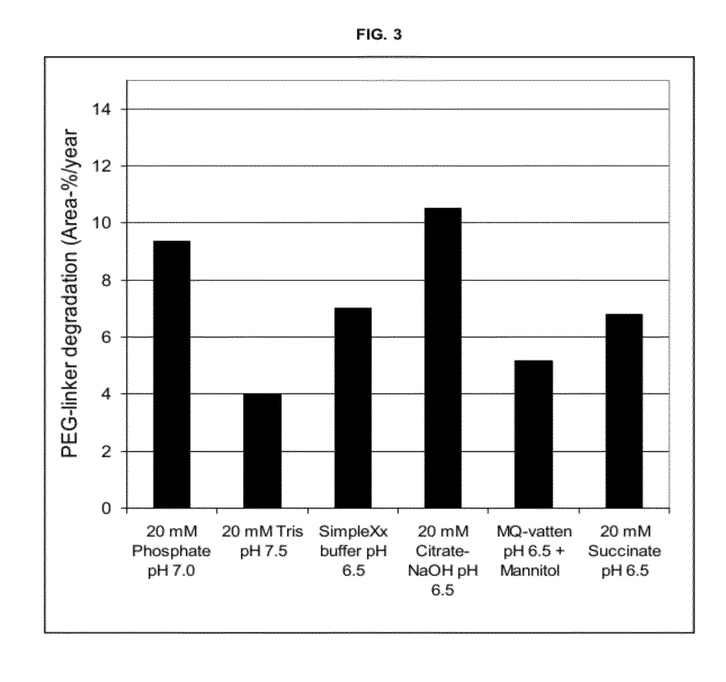

Plotting rate constants in an Arrhenius plot gave good correlation with R-squared value over 0.998 for all buffers. The obtained activation energy and preexponential factor from FIG. 2 was used to calculate degradation rate constants at 5° C. The extent of degradation in one year at 5° C. is shown in FIG. 3. The most stable formulation was Tris-HCl pH 7.5, where 4% was calculated to degrade in one year. Mannitol in MQ-water showed a degradation of 5% in one year.

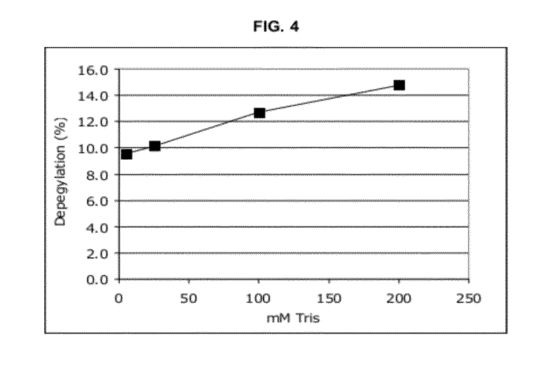

The effect of buffer concentration was studied with 3 mg/ml PEG-(Gln-141) hGH. The peptide was incubated with different concentrations of Tris-HCl at pH 7.5 (+0.5% phenol) at 40° C. for 13 days. The results (FIG. 4) shows that the stability increases with decreasing concentration of the buffer.

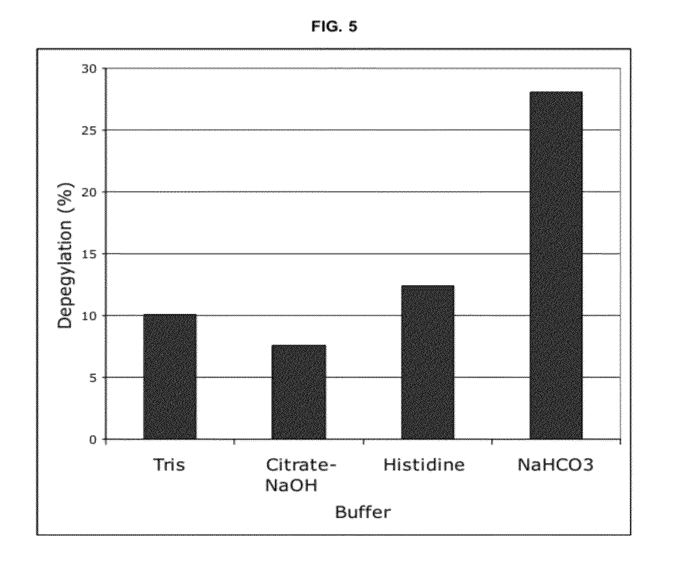

In yet another screening study 3 mg/ml PEG-(Gln-141) hGH was incubated in 4 different 25 mM buffers at pH 7.5 (+0.5% phenol) for one week at 40° C. The amount of depegylation is shown in FIG. 5, again demonstrating the effect of buffer species. Citrate-NaOH buffer and Tris at pH 7.5 showed the highest conjugation stability.

The effect of long term storage was evaluated in Tris and Citrate buffers (5 and 20 mM). The amount of de-PEGylated growth hormone was determined by SEC-HPLC after storage at 5° C., 25° C. or 40° C. as indicated in the tables below. A pharmaceutical composition including PEG-(Gln-141) hGH 6.8 mg/ml and a preservative was used for the study. A concentration of 0.4% phenol was used when testing the pH interval 7.0-8.0 (Table 3 and 4) and a phenol concentration of 0.55% was use when testing the pH interval 6.4-7.0 (Table 2). All compositions additionally included mannitol at a concentration from 35 mg/ml to 45 mg/ml.

TABLE 2
The amount of de-PEGylated hGH meausured after 2 months storage at 40°
C., 3 months storage at 25° C., 6 and 24 months of storage at 5° C.

| | | DePEGylate hGH | | | | | | | | |
|---|---|---|---|---|---|---|---|---|---|---|
| | | | 5° C. | | 5° C. | | 25° C. | | 40° C. | |
| | | T = 0 | (6 months storage) | | (24 months storage) | | (3 months storage) | | (2 months storage) | |
| | | % total | % total | % depegyla- | % total | % depegyla- | % total | % depegyla- | % total | % depegyla- |
| | | depegyla- | depegyla- | tion/ | depegyla- | tion/ | depegyla- | tion/ | depegyla- | tion/ |
| Buffer | pH | tion | tion | month | tion | month | tion | month | tion | month |
| 5 mM | 6.4 | 0.6 | 3.0 | 0.41 | 5.1 | 0.18 | 6.1 | 1.72 | NA | NA |
| Tris | | | | | | | | | | |
| 5 mM | 6.6 | 0.5 | 2.4 | 0.33 | 4.6 | 0.17 | 5.7 | 1.67 | NA | NA |
| Tris | | | | | | | | | | |
| 5 mM | 6.8 | 0.3 | 1.8 | 0.25 | 4.3 | 0.16 | 5.5 | 1.68 | NA | NA |
| Tris | | | | | | | | | | |
| 5 mM | 7.0 | 0.3 | 1.4 | 0.19 | 3.7 | 0.15 | 5.4 | 1.69 | NA | NA |
| Tris | | | | | | | | | | |

% de-PEGylation per months is calculated by by linear regression making a trendline. Tested compositions include 39.9 mg/ml mannitol. NA: Not analysed

TABLE 3
The amount of de-PEGylated hGH meausured after 2 months storage at 40°
C., 3 months storage at 25° C., 6 and 24 months of storage at 5° C.

| | | DePEGylate hGH | | | | | | | | |
|---|---|---|---|---|---|---|---|---|---|---|
| | | | 5° C. | | 5° C. | | 25° C. | | 40° C. | |
| | | T = 0 | (6 months storage) | | (24 months storage) | | (3 months storage) | | (2 months storage) | |
| | | % total | % total | % depegyla- | % total | % depegyla- | % total | % depegyla- | % total | % depegyla- |
| | | depegyla- | depegyla- | tion/ | depegyla- | tion/ | depegyla- | tion/ | depegyla- | tion/ |
| Buffer | pH | tion | tion | month | tion | month | tion | month | tion | month |
| 5 mM | 7.0 | 0.1 | 1.4 | 0.23 | 4.0 | 0.16 | 5.9 | 1.90 | 9.7 | 4.45 |
| Tris | | | | | | | | | | |
| 5 mM | 7.2 | 0.1 | 1.2 | 0.19 | 3.3 | 0.14 | 5.3 | 1.71 | 10.0 | 4.63 |
| Tris | | | | | | | | | | |
| 5 mM | 7.4 | 0.1 | 0.9 | 0.13 | 2.9 | 0.12 | 5.2 | 1.69 | 11.0 | 5.17 |
| Tris | | | | | | | | | | |
| 5 mM | 7.6 | 0.1 | 0.9 | 0.14 | 2.7 | 0.11 | 4.9 | 1.60 | 11.2 | 5.49 |
| Tris | | | | | | | | | | |
| 5 mM | 7.8 | 0.1 | 0.8 | 0.12 | 2.5 | 0.10 | 5.0 | 1.62 | 11.4 | 5.40 |
| Tris | | | | | | | | | | |
| 5 mM | 8.0 | 0.1 | 0.8 | 0.12 | 2.5 | 0.10 | 4.9 | 1.58 | 12.9 | 6.21 |
| Tris | | | | | | | | | | |
| 20 mM | 7.0 | 0.2 | 1.4 | 0.22 | 4.2 | 0.17 | 6.2 | 2.00 | 9.9 | 4.49 |
| Tris | | | | | | | | | | |
| 20 mM | 7.2 | 0.1 | 1.1 | 0.17 | 3.4 | 0.14 | 6.1 | 2.01 | 10.5 | 4.72 |
| Tris | | | | | | | | | | |
| 20 mM | 7.4 | 0.1 | 0.9 | 0.14 | 3.1 | 0.13 | 5.7 | 1.86 | 12.1 | 5.56 |
| Tris | | | | | | | | | | |
| 20 mM | 7.6 | 0.1 | 0.9 | 0.14 | 2.9 | 0.12 | 5.7 | 1.87 | 12.7 | 6.01 |
| Tris | | | | | | | | | | |
| 20 mM | 7.8 | 0.1 | 0.9 | 0.13 | 2.9 | 0.12 | 5.6 | 1.84 | 13.4 | 6.38 |
| Tris | | | | | | | | | | |
| 20 mM | 8.0 | 0.1 | 1.0 | 0.15 | 3.1 | 0.13 | 5.7 | 1.88 | 14.2 | 6.83 |
| Tris | | | | | | | | | | |

% de-PEGylation per months is calculated by by linear regression making a trendline. Tested compositions with 5 mM tris include 42.6 mg/ml mannitol. Tested compositions with 20 mM tris include 42.1 mg/ml mannitol. NA: Not analysed.

TABLE 4
The amount of de-PEGylated hGH meausured after 2 months storage at 40°
C., 3 months storage at 25° C., 3 and 6 months of storage at 5° C.

| | | De-PEGylated hGH | | | | | | | | |
|---|---|---|---|---|---|---|---|---|---|---|
| | | | 5° C. | | 5° C. | | 25° C. | | 40° C. | |
| | | T = 0 | (3 months storage) | | (6 months storage) | | (3 months storage) | | (2 months storage) | |
| | | % total | % total | % depegyla- | % total | % depegyla- | % total | % depegyla- | % total | % depegyla- |
| | | depegyla- | depegyla- | tion/ | depegyla- | tion/ | depegyla- | tion/ | depegyla- | tion/ |
| Buffer | pH | tion | tion | month | tion | month | tion | month | tion | month |
| 5 mM | 7.0 | 0.2 | 1.1 | 0.30 | 1.9 | 0.30 | 5.5 | 1.75 | 9.8 | 4.55 |
| Citrate | | | | | | | | | | |
| 5 mM | 7.2 | 0.1 | 0.8 | 0.22 | 1.5 | 0.24 | 5.3 | 1.70 | 10.5 | 4.95 |
| Citrate | | | | | | | | | | |
| 5 mM | 7.4 | 0.1 | 0.6 | 0.16 | 1.1 | 0.17 | 5.0 | 1.62 | 10.7 | 5.07 |
| Citrate | | | | | | | | | | |
| 5 mM | 7.6 | 0.1 | 0.5 | 0.12 | 0.9 | 0.13 | 4.8 | 1.56 | 11.1 | 5.27 |
| Citrate | | | | | | | | | | |
| 5 mM | 7.8 | 0 | 0.5 | 0.15 | 0.9 | 0.14 | 4.7 | 1.55 | 11.7 | 5.63 |
| Citrate | | | | | | | | | | |
| 5 mM | 8.0 | 0.1 | 0.4 | 0.10 | 0.8 | 0.13 | 4.9 | 1.60 | 13.0 | 6.29 |
| Citrate | | | | | | | | | | |
| 20 mM | 7.0 | 0.1 | 1.4 | 0.42 | 2.5 | 0.42 | 6.5 | 2.08 | 11.0 | 5.14 |
| Citrate | | | | | | | | | | |
| 20 mM | 7.2 | 0.2 | 1.0 | 0.26 | 1.8 | 0.28 | 5.6 | 1.78 | 11.4 | 5.33 |
| Citrate | | | | | | | | | | |
| 20 mM | 7.4 | 0.1 | 0.7 | 0.19 | 1.2 | 0.19 | 5.1 | 1.66 | 11.8 | 5.63 |
| Citrate | | | | | | | | | | |
| 20 mM | 7.6 | 0.1 | 0.5 | 0.12 | 1.0 | 0.15 | 5.0 | 1.61 | 12.8 | 6.13 |
| Citrate | | | | | | | | | | |
| 20 mM | 7.8 | 0.1 | 0.5 | 0.12 | 0.9 | 0.13 | 5.1 | 1.65 | 13.2 | 6.33 |
| Citrate | | | | | | | | | | |
| 20 mM | 8.0 | 0.1 | 0.5 | 0.12 | 0.9 | 0.13 | 5.5 | 1.81 | 14.3 | 6.92 |
| Citrate | | | | | | | | | | |

% de-PEGylation per months is calculated by by linear regression making a trendline. Tested compositions with 5 mM citrat include 42.5 mg/ml mannitol. Tested compositions with 20 mM citrat include 41.6 mg/ml mannitol.

### Example 3

### Determination of Growth Hormone Activity, Assay I

BA/F3 cells are originally IL-3 dependent for growth and survival. IL-3 activates JAK-2 and STAT which are the same mediators GH is activating upon stimulation. After transfection of the hGH receptor (GHR) the cell line is transformed into a growth hormone-dependent cell line. This clone can be used to evaluate the effect of different growth hormone samples on the survival of the BAF-3 GHR.

The BA/F3-GHR cells are grown in starvation medium (culture medium without growth hormone) for 24 h at 37° C., 5% CO₂.

The cells are washed and resuspended in starvation medium and seeded in plates. Growth hormone compound or hGH, in different concentrations, or control is added to the cells, and the plates are incubated for three days at 37° C., 5% CO₂.

AlamarBlue® is added to each well and the cells are incubated for further 4 h. AlamarBlue® is a redox indicator, which is reduced by reactions of cellular metabolism and, therefore, provides an indirect measure of viable cell number and cell growth.

The metabolic activity of the cells was measured using a fluorescence plate reader. A software based on 4 Parameter Logistic curve fitting was used to determine specific potency of the growth hormone compound.

1. An aqueous pharmaceutical composition comprising a therapeutic peptide derivatized with a property-modifying group, wherein the property-modifying group is conjugated to the peptide in a position different from the N-terminal amino acid of the peptide by use of a linker comprising an oxime bond, wherein the pH of the composition is above 7.

2. An aqueous pharmaceutical composition according to claim 1, comprising a buffer selected from the group consisting of citrate-KOH, Tris-HNO₃, Tris-H₂PO₄, citrate-NaOH or Tris-HCl.

3. An aqueous pharmaceutical composition according to claim 2, wherein the buffer is citrate-NaOH or Tris-HCl.

4. An aqueous pharmaceutical composition according to claim 1, comprising a buffer, wherein the buffer is present in a concentration of 100 mM or less.

5. An aqueous pharmaceutical composition according to claim 4, wherein the buffer is present in a concentration of from 1 mM to 100 mM.

6. An aqueous pharmaceutical composition according to claim 1, wherein the pharmaceutical composition further comprises a preservative.

7. An aqueous pharmaceutical composition according to claim 5, wherein said preservative is phenol.

8. A pharmaceutical composition according to claim 1, wherein the pharmaceutical composition is isotonic.

9. A pharmaceutical composition according claim 8, wherein said isotonicity is provided by the presence of an isotonic agent, wherein said isotonic agent is mannitol.

10. A pharmaceutical composition according to claim 1, wherein the pH of the composition is from about 7.2 to about 8.

11. A pharmaceutical composition according to claim 1, wherein the property-modifying group is conjugated to the peptide in a position different from the C-terminal amino acid.

12. A pharmaceutical composition according to claim 1, wherein the derivatized therapeutic peptide is a derivatized growth hormone compound.

13. A pharmaceutical composition according to claim 12, wherein the property-modifying group is conjugated to the growth hormone compound in a position corresponding to Gln40 and/or Gln141 in human growth hormone.

14. A pharmaceutical composition according to claim 1, wherein the derivatized therapeutic peptide is selected from the group consisting of:
N^{δ141/40}-2-(O-(4-(4-(1,3-bis(mPEG(10k)ylaminocarbonyloxy)prop-2-yloxy)butyryl)aminobutyl)-oximino)ethyl hGH;
N^{δ141/40}-2-(O-(4-(4-(mPEG(10k)yloxy)butyryl)aminobutyl)oximino)ethyl hGH;
N^{δ141/40}-2-(O-(4-(3-(mPEG(10k)yloxy)propionyl)aminobutyl)oximino)ethyl hGH;
N^{δ141/40}-2-(O-(4-(4-(2,3-bis(mPEG(10k)yloxy)prop-1-yloxy)butyryl)aminobutyl)oximino)ethyl hGH;
N^{δ141/40}-2-O-(4-(5-(mPEG(10k)yloxy-5-oxopentanoyl)aminobutyl)oximino)ethyl hGH;
N^{δ141/40}-3-((4-(2-(2-(2-(2-(4-(1,3-bis(mPEG(10k)ylaminocarbonyloxy)prop-2-yloxy)-butyrylamino)ethoxy)ethoxy)ethoxy)ethoxy)butylidene)aminoxy)prop-1-yloxy hGH;
N^{δ141/40}-3-((4-(1,3-bis(mPEG(10k)ylaminocarbonyloxy)prop-2-yloxy)butylidene)-aminoxy)propyloxy hGH;
N^{δ141/40}-3-((4-(mPEG(10k)yloxy)butylidene)aminoxy)propyloxy hGH;
N^{δ141/40}-3-((4-(2,3-bis(mPEG(10k)yloxy)prop-1-yloxy)butylidene)aminoxy)propyloxy hGH;
N^{δ141/40}-3-((3-(mPEG(10k)yloxy)propylidene)aminoxy)propyloxy hGH;
N^{δ141/40}-2-(O-(2-(3-(2,3-bis(mPEG(10k)yloxy)propyloxy)propylamino)-2-oxoethyl)oximino)ethyl hGH;
N^{δ141/40}-3-((4-(2,3-bis(mPEG(10k)yloxy)prop-1-yloxy)propylidene)aminoxy)propyloxy hGH;
N^{δ141/40}-2-(O-(4-(4-(1,3-bis(mPEG(20k)ylaminocarbonyloxy)prop-2-yloxy)butyryl)aminobutyl)-oximino)ethyl hGH;
N^{δ141/40}-2-O-(4-(3-(mPEG(20k)yloxy)propionyl)aminobutyl)oximino)ethyl hGH;
N^{δ141/40}-2-(O-(4-(4-(2,3-bis(mPEG(20k)yloxy)prop-1-yloxy)butyryl)aminobutyl)oximino)ethyl hGH;
N^{δ141/40}-2-(O-(4-(5-(mPEG(20k)yloxy-5-oxopentanoyl)aminobutyl)oximino)ethyl hGH;
N^{δ141/40}-3-((4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene)aminoxy)prop-1-yloxy hGH;
N^{δ141/40}-3-((4-(1,3-bis(mPEG(20k)ylaminocarbonyloxy)prop-2-yloxy)butylidene)-aminoxy)propyloxy hGH;
N^{δ141/40}-3-((4-(mPEG(20k)yloxy)butylidene)aminoxy)propyloxy hGH;
N^{δ141/40}-3-((4-(2,3-bis(mPEG(20k)yloxy)prop-1-yloxy)butylidene)aminoxy)propyloxy hGH;
N^{δ141/40}-3-((3-(mPEG(20k)yloxy)propylidene)aminoxy)propyloxy hGH;
N^{δ141/40}-2-(O-(2-(3-(2,3-bis(mPEG(20k)yloxy)propyloxy)propylamino)-2-oxoethyl)oximino)ethyl hGH;
N^{δ141/40}-3-((4-(2,3-bis(mPEG(20k)yloxy)prop-1-yloxy)propylidene)aminoxy)propyloxy hGH;
N^{δ141/40}-2-(O-(4-(4-(1,3-bis(mPEG(30k)ylaminocarbonyloxy)prop-2-yloxy)butyryl)aminobutyl)-oximino)ethyl hGH;
N^{δ141/40}-2-(O-(4-(3-(mPEG(30k)yloxy)propionyl)aminobutyl)oximino)ethyl hGH;
N^{δ141/40}-2-(O-(4-(4-(2,3-bis(mPEG(30k)yloxy)prop-1-yloxy)butyryl)aminobutyl)oximino)ethyl hGH;
N^{δ141/40}-2-(O-(4-(5-(mPEG(30k)yloxy-5-oxopentanoyl)aminobutyl)oximino)ethyl hGH;
N^{δ141/40}-3-((4-(2-(2-(2-(2-(4-(1,3-bis(mPEG(30k)ylaminocarbonyloxy)prop-2-yloxy)-butyrylamino)ethoxy)ethoxy)ethoxy)ethoxy)butylidene)aminoxy)prop-1-yloxy hGH;
N^{δ141/40}-3-((4-(1,3-bis(mPEG(30k)ylaminocarbonyloxy)prop-2-yloxy)butylidene)-aminoxy)propyloxy hGH;
N^{δ141/40}-3-((4-(mPEG(30k)yloxy)butylidene)aminoxy)propyloxy hGH;
N^{δ141/40}-3-((4-(2,3-bis(mPEG(30k)yloxy)prop-1-yloxy)butylidene)aminoxy)propyloxy hGH;
N^{δ141/40}-3-((3-(mPEG(30k)yloxy)propylidene)aminoxy)propyloxy hGH;
N^{δ141/40}-2-(O-(2-(3-(2,3-bis(mPEG(30k)yloxy)propyloxy)propylamino)-2-oxoethyl)oximino)ethyl hGH;
N^{δ141/40}-3-((4-(2,3-bis(mPEG(30k)yloxy)prop-1-yloxy)propylidene)aminoxy)propyloxy hGH;
N^{δ141/40}-3-((4-((2,3-bis(mPEG(20k)yl)prop-1-yloxy)PEGyloxy)butylidene)aminoxy)propyloxy hGH;
N^{δ141/40}-2-((4-(4-((2,3-bis(mPEG(20k)yl)propyl)PEGyloxy)butyrylamino)butyl)oximino)ethyl hGH;
N^{δ141}-2-(O-(4-(4-(1,3-bis(mPEG(10k)ylaminocarbonyloxy)prop-2-yloxy)butyryl)-amino-butyl)oximino)ethyl hGH;
N^{δ141}-2-(O-(4-(4-(mPEG(10k)yloxy)butyryl)aminobutyl)oximino)ethyl hGH;
N^{δ141}-2-(O-(4-(3-(mPEG(10k)yloxy)propionyl)aminobutyl)oximino)ethyl hGH;
N^{δ141}-2-(O-(4-(4-(2,3-bis(mPEG(10k)yloxy)prop-1-yloxy)butyryl)aminobutyl)oximino)ethyl hGH;
N^{δ141}-2-(O-(4-(5-(mPEG(10k)yloxy-5-oxopentanoyl)aminobutyl)oximino)ethyl hGH;
N^{δ141}-3-((4-(2-(2-(2-(2-(4-(1,3-bis(mPEG(10k)ylaminocarbonyloxy)prop-2-yloxy)-butyrylamino)ethoxy)ethoxy)ethoxy)ethoxy)butylidene)aminoxy)prop-1-yloxy hGH;
N^{δ141}-3-((4-(1,3-bis(mPEG(10k)ylaminocarbonyloxy)prop-2-yloxy)butylidene)-aminoxy)propyloxy hGH;
N^{δ141}-3-((4-(mPEG(10k)yloxy)butylidene)aminoxy)propyloxy hGH;
N^{δ141}-3-((4-(2,3-bis(mPEG(10k)yloxy)prop-1-yloxy)butylidene)aminoxy)propyloxy hGH;
N^{δ141}-3-((3-(mPEG(10k)yloxy)propylidene)aminoxy)propyloxy hGH;
N^{δ141}-2-(O-(2-(3-(2,3-bis(mPEG(10k)yloxy)propyloxy)propylamino)-2-oxoethyl)oximino)ethyl hGH;
N^{δ141}-3-((4-(2,3-bis(mPEG(10k)yloxy)prop-1-yloxy)propylidene)aminoxy)propyloxy hGH;
N^{δ141}-2-(O-(4-(4-(1,3-bis(mPEG(20k)ylaminocarbonyloxy)prop-2-yloxy)butyryl)-minobutyl)oximino)ethyl hGH;
N^{δ141}-2-(O-(4-(3-(mPEG(20k)yloxy)propionyl)aminobutyl)oximino)ethyl hGH;
N^{δ141}-2-(O-(4-(4-(2,3-bis(mPEG(20k)yloxy)prop-1-yloxy)butyryl)aminobutyl)oximino)ethyl hGH;
N^{δ141}-2-(O-(4-(5-(mPEG(20k)yloxy-5-oxopentanoyl)aminobutyl)oximino)ethyl hGH;
N^{δ141}-3-((4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene)aminoxy)prop-1-yloxy hGH;
N^{δ141}-3-((4-(1,3-bis(mPEG(20k)ylaminocarbonyloxy)prop-2-yloxy)butylidene)-aminoxy)propyloxy hGH;
N^{δ141}-3-((4-(mPEG(20k)yloxy)butylidene)aminoxy)propyloxy hGH;
N^{δ141}-3-((4-(2,3-bis(mPEG(20k)yloxy)prop-1-yloxy)butylidene)aminoxy)propyloxy hGH;
N^{δ141}-3-((3-(mPEG(20k)yloxy)propylidene)aminoxy)propyloxy hGH;
N^{δ141}-2-(O-(2-(3-(2,3-bis(mPEG(20k)yloxy)propyloxy)propylamino)-2-oxoethyl)oximino)ethyl hGH;
N^{δ141}-3-((4-(2,3-bis(mPEG(20k)yloxy)prop-1-yloxy)propylidene)aminoxy)propyloxy hGH;
N^{δ141}-2-(O-(4-(4-(1,3-bis(mPEG(30k)ylaminocarbonyloxy)prop-2-yloxy)butyryl)-aminobutyl)oximino)ethyl hGH;
N^{δ141}-2-(O-(4-(3-(mPEG(30k)yloxy)propionyl)aminobutyl)oximino)ethyl hGH;
N^{δ141}-2-(O-(4-(4-(2,3-bis(mPEG(30k)yloxy)prop-1-yloxy)butyryl)aminobutyl)oximino)ethyl hGH;
N^{δ141}-2-(O-(4-(5-(mPEG(30k)yloxy-5-oxopentanoyl)aminobutyl)oximino)ethyl hGH;
N^{δ141}-3-((4-(2-(2-(2-(2-(4-(1,3-bis(mPEG(30k)ylaminocarbonyloxy)prop-2-yloxy)-butyrylamino)ethoxy)ethoxy)ethoxy)ethoxy)butylidene)aminoxy)prop-1-yloxy hGH;
N^{δ141}-3-((4-(1,3-bis(mPEG(30k)ylaminocarbonyloxy)prop-2-yloxy)butylidene)-aminoxy)propyloxy hGH;
N^{δ141}-3-((4-(mPEG(30k)yloxy)butylidene)aminoxy)propyloxy hGH;
N^{δ141}-3-((4-(2,3-bis(mPEG(30k)yloxy)prop-1-yloxy)butylidene)aminoxy)propyloxy hGH;
N^{δ141}-3-((3-(mPEG(30k)yloxy)propylidene)aminoxy)propyloxy hGH;
N^{δ141}-2-(O-(2-(3-(2,3-bis(mPEG(30k)yloxy)propyloxy)propylamino)-2-oxoethyl)oximino)ethyl hGH;
N^{δ141}-3-((4-(2,3-bis(mPEG(30k)yloxy)prop-1-yloxy)propylidene)aminoxy)propyloxy hGH;
N^{δ141}-3-((4-((2,3-bis(mPEG(20k)yloxy)prop-1-yl)PEGyloxy)butylidene)aminoxy)propyloxy hGH;
N^{δ141}-2-((4-(4-((2,3-bis(mPEG(20k)yl)propyl)PEGyloxy)butyrylamino)butyl)oximino)ethyl hGH;
N^{δ40}-2-(O-(4-(4-(1,3-bis(mPEG(10k)ylaminocarbonyloxy)prop-2-yloxy)butyryl)-amino-butyl)oximino)ethyl hGH;
N^{δ40}-2-(O-(4-(4-(mPEG(10k)yloxy)butyryl)aminobutyl)oximino)ethyl hGH;
N^{δ40}-2-(O-(4-(3-(mPEG(10k)yloxy)propionyl)aminobutyl)oximino)ethyl hGH;
N^{δ40}-2-(O-(4-(4-(2,3-bis(mPEG(10k)yloxy)prop-1-yloxy)butyryl)aminobutyl)oximino)ethyl hGH;
N^{δ40}-2-(O-(4-(5-(mPEG(10k)yloxy-5-oxopentanoyl)aminobutyl)oximino)ethyl hGH;
N^{δ40}-3-((4-(2-(2-(2-(2-(4-(1,3-bis(mPEG(10k)ylaminocarbonyloxy)prop-2-yloxy)butyryl-amino)ethoxy)ethoxy)ethoxy)ethoxy)butylidene)aminoxy)prop-1-yloxy hGH;
N^{δ40}-3-((4-(1,3-bis(mPEG(10k)ylaminocarbonyloxy)prop-2-yloxy)butylidene)-aminoxy)-propyloxy hGH;
N^{δ40}-3-((4-(mPEG(10k)yloxy)butylidene)aminoxy)propyloxy hGH;
N^{δ40}-3-((4-(2,3-bis(mPEG(10k)yloxy)prop-1-yloxy)butylidene)aminoxy)propyloxy hGH;
N^{δ40}-3-((3-(mPEG(10k)yloxy)propylidene)aminoxy)propyloxy hGH;
N^{δ40}-2-(O-(2-(3-(2,3-bis(mPEG(10k)yloxy)propyloxy)propylamino)-2-oxoethyl)oximino)ethyl hGH;
N^{δ40}-3-((4-(2,3-bis(mPEG(10k)yloxy)prop-1-yloxy)propylidene)aminoxy)propyloxy hGH;
N^{δ40}-2-(O-(4-(4-(1,3-bis(mPEG(20k)ylaminocarbonyloxy)prop-2-yloxy)butyryl)-aminobutyl)oximino)ethyl hGH;
N^{δ40}-2-(O-(4-(3-(mPEG(20k)yloxy)propionyl)aminobutyl)oximino)ethyl hGH;
N^{δ40}-2-(O-(4-(4-(2,3-bis(mPEG(20k)yloxy)prop-1-yloxy)butyryl)aminobutyl)oximino)ethyl hGH;
N^{δ40}-2-(O-(4-(5-(mPEG(20k)yloxy-5-oxopentanoyl)aminobutyl)oximino)ethyl hGH;
N^{δ40}-3-((4-(2-(2-(2-(2-(4-(1,3-bis(mPEG(20k)ylaminocarbonyloxy)prop-2-yloxy)butyryl-amino)ethoxy)ethoxy)ethoxy)ethoxy)butylidene)aminoxy)prop-1-yloxy hGH;
N^{δ40}-3-((4-(1,3-bis(mPEG(20k)ylaminocarbonyloxy)prop-2-yloxy)butylidene)-aminoxy)-propyloxy hGH;
N^{δ40}-3-((4-(mPEG(20k)yloxy)butylidene)aminoxy)propyloxy hGH;
N^{δ40}-3-((4-(2,3-bis(mPEG(20k)yloxy)prop-1-yloxy)butylidene)aminoxy)propyloxy hGH;
N^{δ40}-3-((3-(mPEG(20k)yloxy)propylidene)aminoxy)propyloxy hGH;
N^{δ40}-2-(O-(2-(3-(2,3-bis(mPEG(20k)yloxy)propyloxy)propylamino)-2-oxoethyl)oximino)ethyl hGH;
N^{δ40}-3-((4-(2,3-bis(mPEG(20k)yloxy)prop-1-yloxy)propylidene)aminoxy)propyloxy hGH;
N^{δ40}-2-(O-(4-(4-(1,3-bis(mPEG(30k)ylaminocarbonyloxy)prop-2-yloxy)butyryl)-aminobutyl)oximino)ethyl hGH;
N^{δ40}-2-(O-(4-(3-(mPEG(30k)yloxy)propionyl)aminobutyl)oximino)ethyl hGH;
N^{δ40}-2-(O-(4-(4-(2,3-bis(mPEG(30k)yloxy)prop-1-yloxy)butyryl)aminobutyl)oximino)ethyl hGH;
N^{δ40}-2-(O-(4-(5-(mPEG(30k)yloxy-5-oxopentanoyl)aminobutyl)oximino)ethyl hGH;
N^{δ40}-3-((4-(2-(2-(2-(2-(4-(1,3-bis(mPEG(30k)ylaminocarbonyloxy)prop-2-yloxy)butyryl-amino)ethoxy)ethoxy)ethoxy)ethoxy)butylidene)aminoxy)prop-1-yloxy hGH;
N^{δ40}-3-((4-(1,3-bis(mPEG(30k)ylaminocarbonyloxy)prop-2-yloxy)butylidene)-aminoxy)-propyloxy hGH;
N^{δ40}-3-((4-(mPEG(30k)yloxy)butylidene)aminoxy)propyloxy hGH;
N^{δ40}-3-((4-(2,3-bis(mPEG(30k)yloxy)prop-1-yloxy)butylidene)aminoxy)propyloxy hGH;
N^{δ40}-3-((3-(mPEG(30k)yloxy)propylidene)aminoxy)propyloxy hGH;
N^{δ40}-2-(O-(2-(3-(2,3-bis(mPEG(30k)yloxy)propyloxy)propylamino)-2-oxoethyl)oximino)ethyl hGH;
N^{δ40}-3-((4-(2,3-bis(mPEG(30k)yloxy)prop-1-yloxy)propylidene)aminoxy)propyloxy hGH;
N^{δ40}-3-((4-((2,3-bis(mPEG(20k)yloxy)prop-1-yl)PEGyloxy)butylidene)aminoxy)propyloxy hGH;
N^{δ40}-2-((4-(4-((2,3-bis(mPEG(20k)yl)propyl)PEGyloxy)butyrylamino)butyl)oximino)ethyl hGH;
N^{ε}141-(2-O-(4-(4-(1,3-bis(mPEG(20K)aminocarbonyloxy)-2-propyloxy)butyrylamino)-butyl)oxyimino)ethyl) hGH;
N^{ε}141-(2-(O-(4-(4-(mPEG(30K)oxy)butyrylamino)-butyl)oxyimino)-ethyl) hGH;
N^{ε}141-(3-((4-(2-(2-(2-(2-(4-(1,3-bis(mPEG(20K)ylaminocarbonyloxy)-2-propyloxy)-butyrylamino)ethoxy)ethoxy)ethoxy)ethoxy)butylidene)aminoxy)propyloxy) hGH;
N^{ε}141-(2-(2-(2,3-bis(mPEG(20K)yloxy)propyloxycarbonylamino)ethyloximino)ethyl) hGH;
N^{ε}141-(2-(O-(2-(2-(mPEG(40K)yloxy)ethylamino)-2-oxoethyl)oximino)ethyl) hGH;
N^{ε}141-(2-(3-(4-((1,3-bis(mPEG(30K)ylaminocarbonyloxy)-2-propyloxy)butylidene)-aminoxy)propyloxyimino)ethyl) hGH;
N^{ε}141/40-(2-O-(4-(4-(1,3-bis(mPEG(20K)aminocarbonyloxy)-2-propyloxy)butyryl-amino)butyl)oxyimino)ethyl) hGH;
N^{ε}141/40-(2-(O-(4-(4-(mPEG(30K)oxy)butyrylamino)-butyl)oxyimino)-ethyl) hGH;
N^{ε}141/40-(3-((4-(2-(2-(2-(2-(4-(1,3-bis(mPEG(20K)ylaminocarbonyloxy)-2-propyloxy)-butyrylamino)ethoxy)ethoxy)ethoxy)ethoxy)butylidene)aminoxy)propyloxy) hGH;
N^{ε}141/40-(2-(2-(2,3-(mPEG(20K)yloxy)propyloxycarbonylamino)ethyloximino)ethyl) hGH;
N^{ε}141/40-(2-(O-(2-(2-(mPEG(40K)yloxy)ethylamino)-2-oxoethyl)oximino)ethyl) hGH;
N^{ε}141/40-(2-(3-(4-((1,3-bis(mPEG(30K)ylaminocarbonyloxy)-2-propyloxy)butylidene)-aminoxy)propyloxyimino)ethyl) hGH;
N^{ε}40-(2-O-(4-(4-(1,3-bis(mPEG(20K)aminocarbonyloxy)-2-propyloxy)butyrylamino)-butyl)oxyimino)ethyl) hGH;
N^{ε}40-(2-(O-(4-(4-(mPEG(30K)oxy)butyrylamino)-butyl)oxyimino)-ethyl) hGH;
N^{ε}40-(3-((4-(2-(2-(2-(2-(4-(1,3-bis(mPEG(20K)ylaminocarbonyloxy)-2-propyloxy)-butyrylamino)ethoxy)ethoxy)ethoxy)ethoxy)butylidene)aminoxy)propyloxy) hGH;
N^{ε}40-(2-(2-(2,3-(mPEG(20K)yloxy)propyloxycarbonylamino)ethyloximino)ethyl) hGH;
N^{ε}40-(2-(O-(2-(2-(mPEG(40K)yloxy)ethylamino)-2-oxoethyl)oximino)ethyl) hGH;
N^{ε}40-(2-(3-(4-((1,3-bis(mPEG(30K)ylaminocarbonyloxy)-2-propyloxy)butylidene)-aminoxy)propyloxyimino)ethyl) hGH;

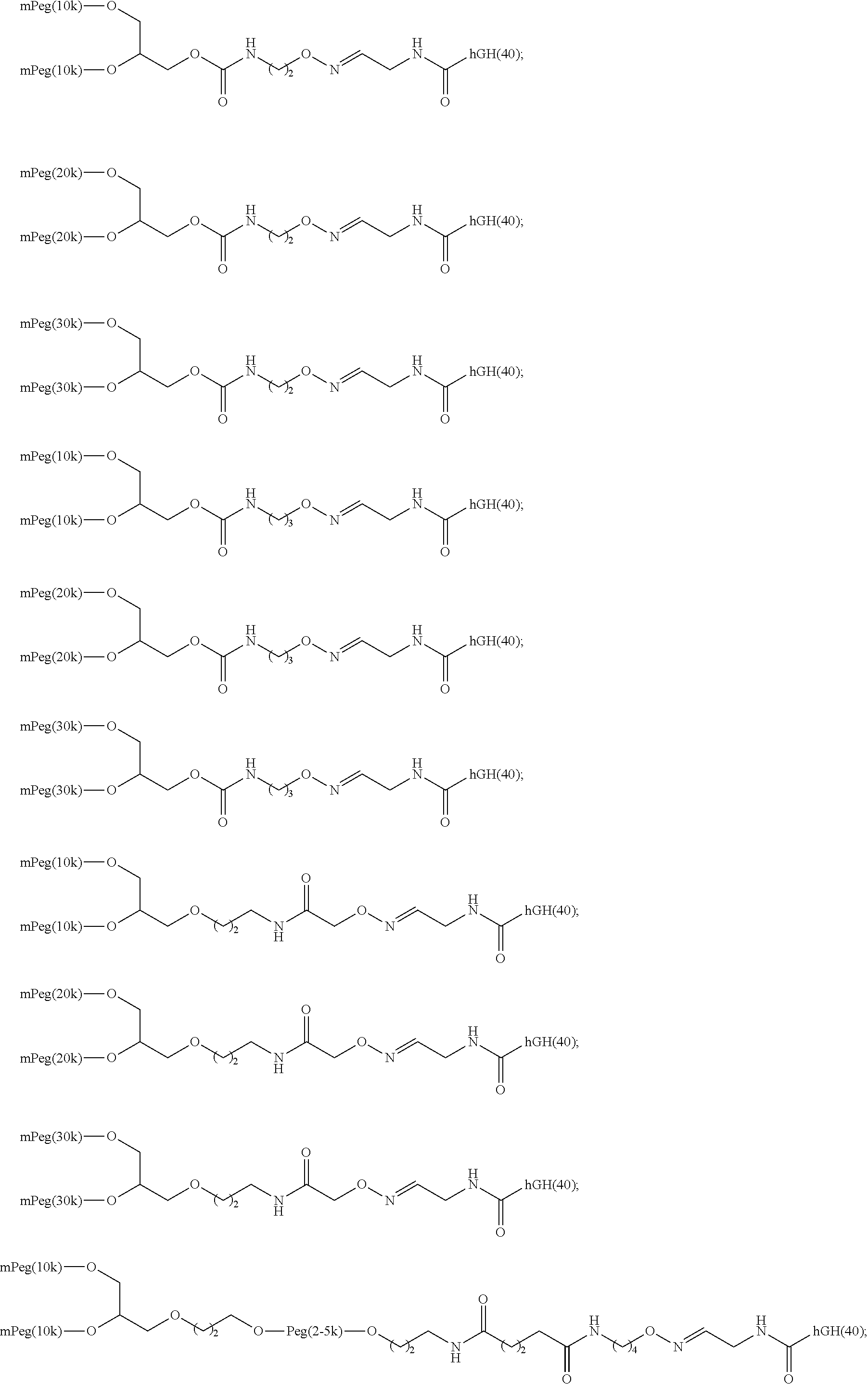

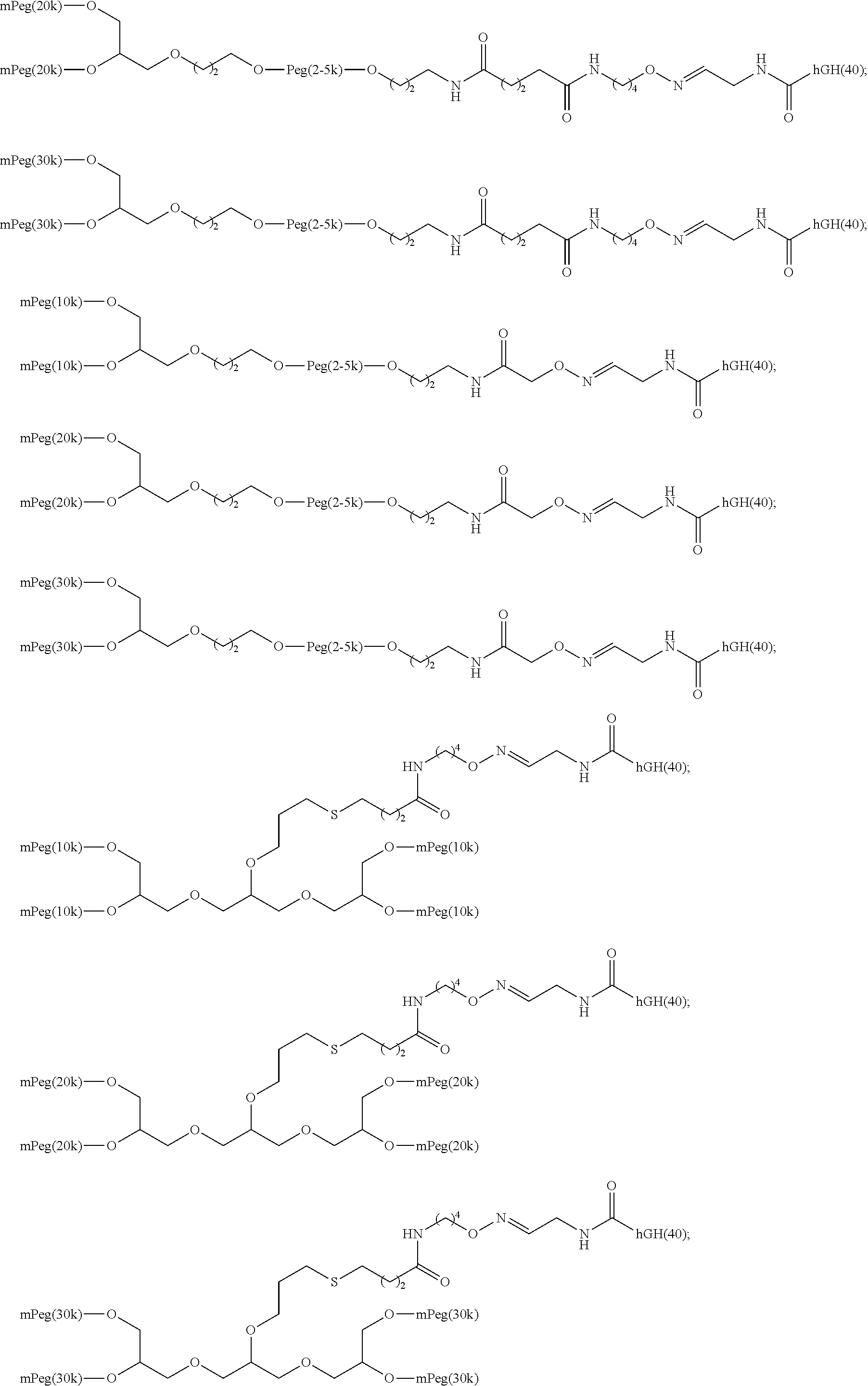

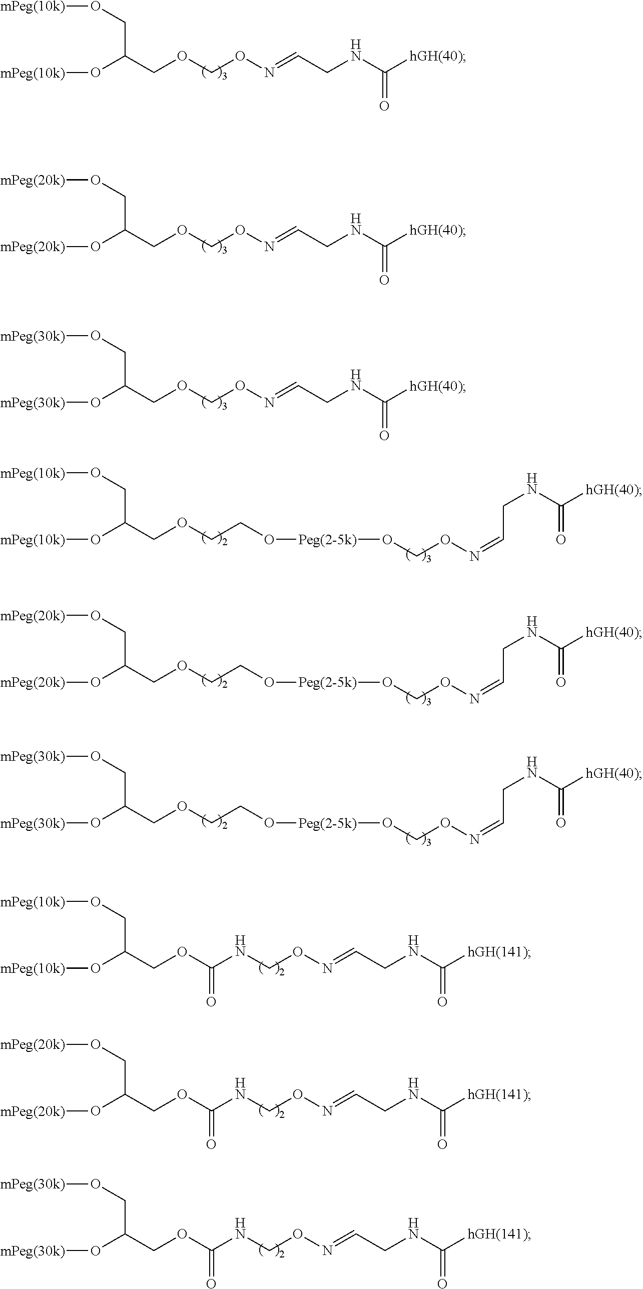

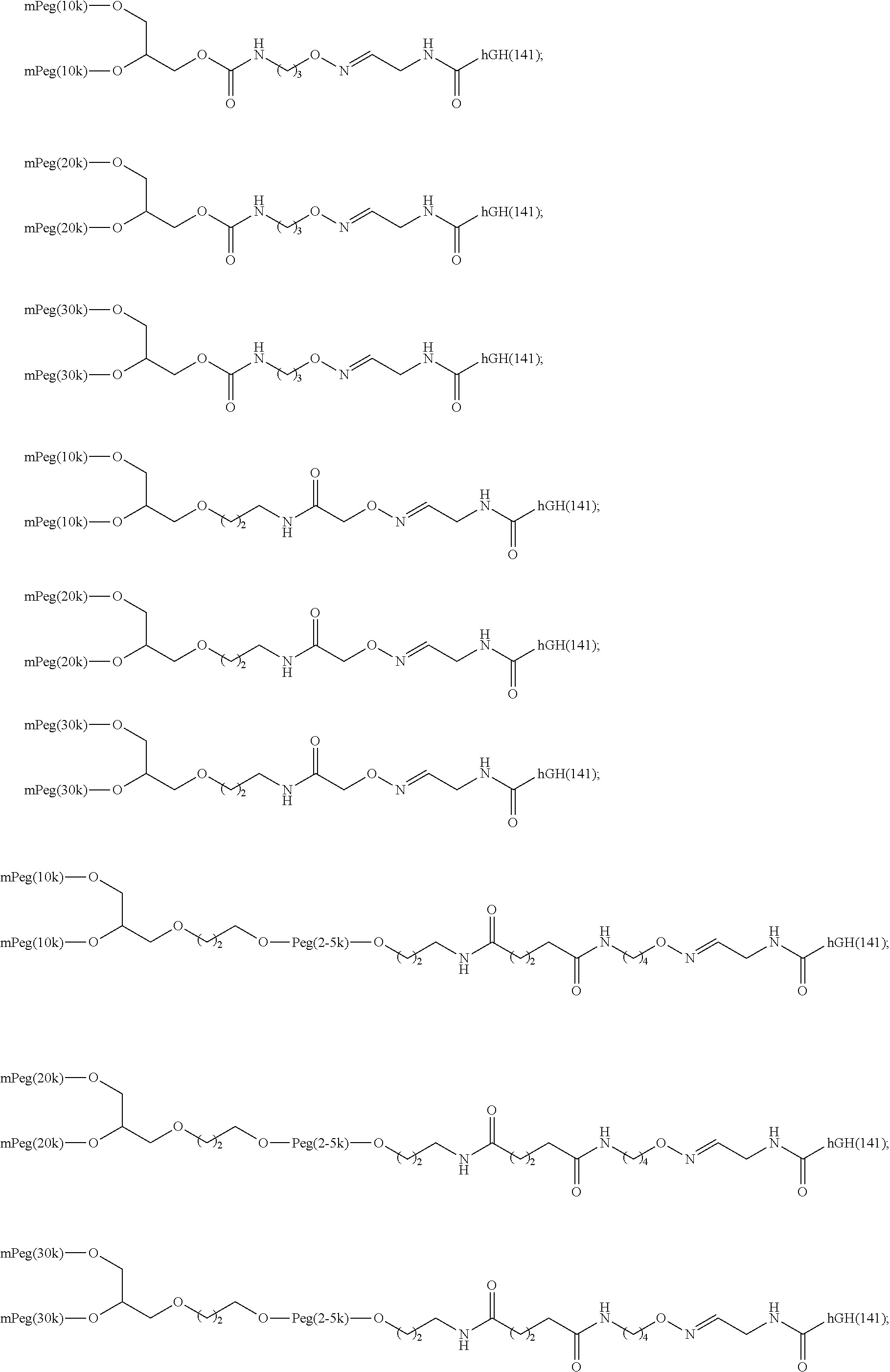

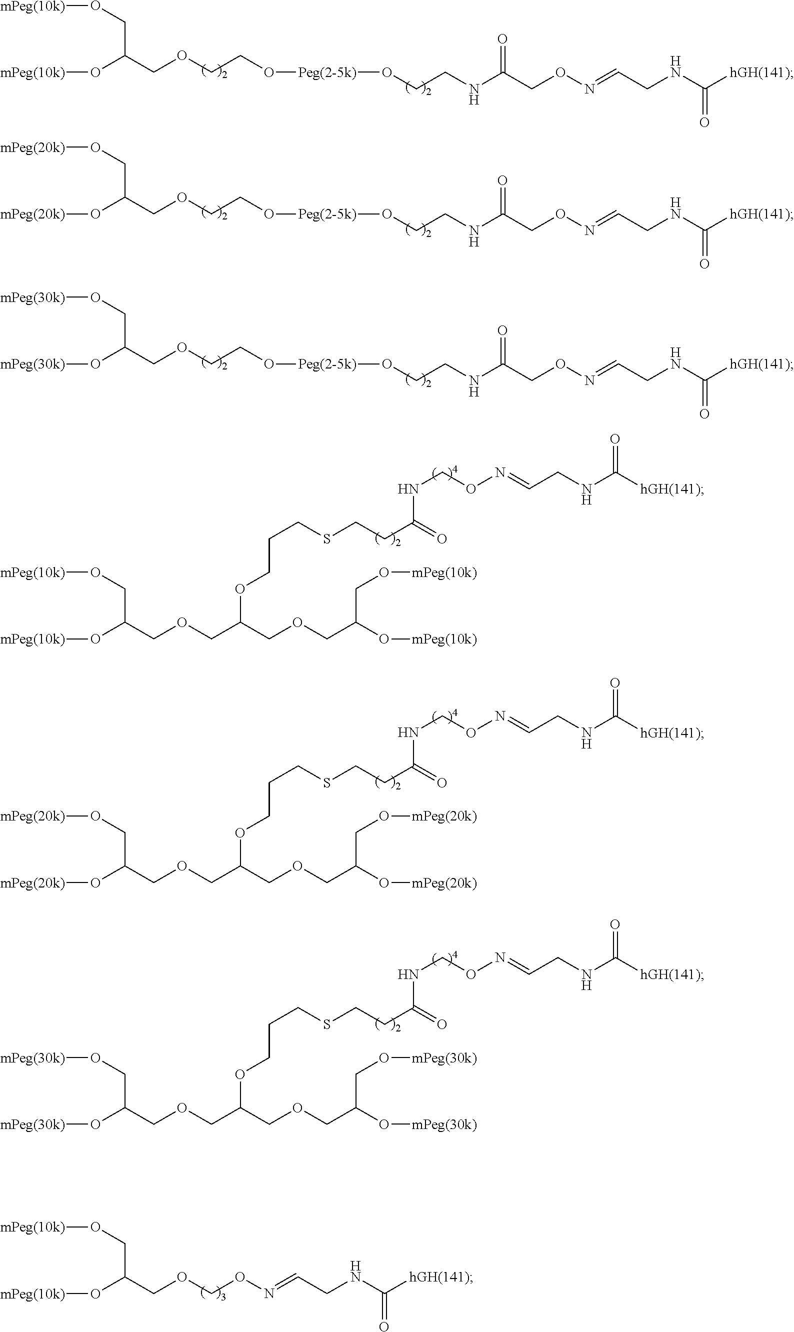

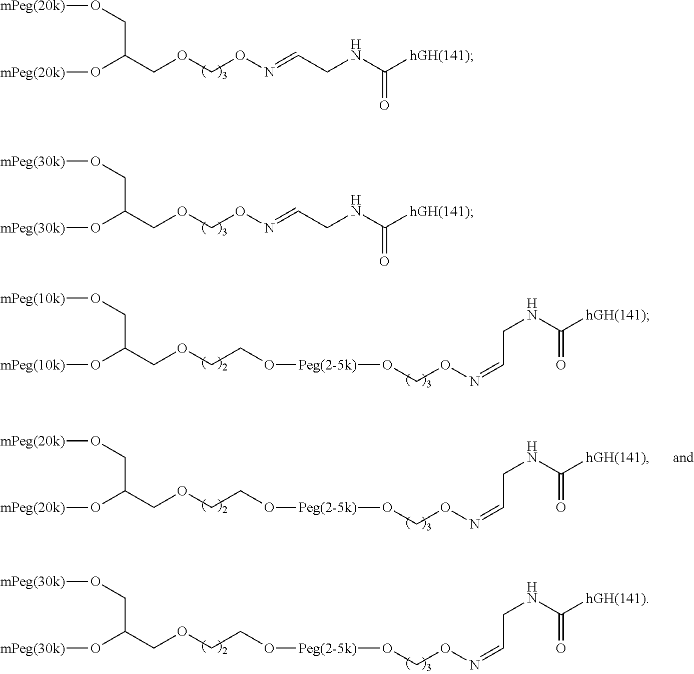

15. A method of treating a patient having a disease that benefits from an increased level of circulating growth hormone comprising administering an effective amount of the pharmaceutical composition of claim 14.

16. A method of treating a patient having a disease that benefits from an increased level of circulating growth hormone comprising administering an effective amount of the a pharmaceutical composition according to claim 13 to a patient in need thereof.


SEQUENCE LISTING (1 sequence)

SEQ ID NO: 1 - protein, 191 aa, Homo sapiens [sequence omitted]